(19) United States
(12) Patent Application Publication
(10) Pub. No.: US 2003/0199530 A1
(43) Pub. Date: Oct. 23, 2003

(54) New compounds derived from quinazoline

(76) Inventors: Solo Goldstein, Suresnes (FR); Alain Dhainaut, Chatou (FR); Andre Tizot, Verrieres Le Buisson (FR); Jean-Luc Fauchere, Saint Cloud (FR); Nathalie Kucharczyk, Fourqueux (FR); John Hickman, Paris (FR); Alain Pierre, Les Alluets Le Roi (FR); Gordon Tucker, Paris (FR); Laurence Kraus-Berthier, Colombes (FR)

Correspondence Address: The Firm of Hueschen and Sage, 500 Columbia Plaza, 350 East Michigan Avenue, Kalamazoo, MI, 49007-3856, US

(21) Appl. No.: 10/390,802

(22) Filed: Mar. 18, 2003

(30) Foreign Application Priority Data
Mar. 18, 2002 (FR) 02 03299

(51) Int. Cl. A61K 31/517; C 07D 4 3/02

(52) U.S. Cl. 514/266.23; 544/284

(57) ABSTRACT

A compound selected from those of formula (I):

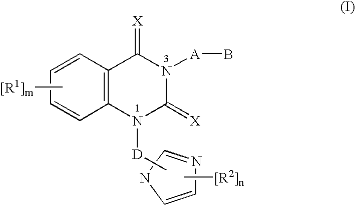

wherein

A, B, D, X, R¹, R², m and n are as defined in the description, their diastereoisomers and addition salts thereof with a pharmaceutically acceptable acid or base.

### FIELD OF THE INVENTION

The compounds of the invention are useful as farnesyl transferase inhibitors.

A large number of proteins are subject to post-translational changes which alter their localisation and their function. In particular, lipid-type modifications allow certain proteins that are inactive in their free form to be anchored in the plasma membrane, which is a crucial step for ensuring their function. This applies to prenylation (Curr. Opin. Cell. Biol., 4, 1992, 1008-1016), which is catalysed by several enzymes: farnesyl transferase (FTase) and the two geranylgeranyl transferases (GGTase-I and GGTase-II) which couple a prenyl group to 15 (trans,trans-famesyl) or 20 (all-trans-geranylgeranyl) carbons on the carboxy terminal moiety of substrate proteins (J. Biol. Chem., 271, 1996, 5289-5292; Curr. Opin. Struct. Biol., 7, 1997, 873-880). FTase catalyses that transfer, starting from farnesyl pyrophosphate, to form a thio ether bond on the cysteine of the terminal tetrapeptide consensus sequence CA₁A₂X found on substrate proteins, C denoting cysteine, A₁ and A₂ denoting an aliphatic amino acid and X denoting a serine, an alanine or a methionine. GGTase-I uses geranylgeranyl pyrophosphate as donor substrate for effecting a similar transfer, but this time the consensus sequence CAAX is terminated by a leucine or a phenylalanine. Those two heterodimeric enzymes share an alpha subunit of 48 kDa, and possess two distinct beta chains, although they have 30% amino acid sequence homology. GGTase-II acts on terminal sequences of the XXCC and XCXC types and has alpha and beta subunits different from those of the afore-mentioned enzymes.

### BACKGROUND OF THE INVENTION

The interest in inhibiting one of those enzymes, FTase, is based on the implication in tumour progression of the prenylated oncogene Ras (Annu. Rev. Biochem., 56, 1987, 779-827). Ras proteins exist in four major forms, Harvey or H-Ras, N-Ras, and Kirsten or K-Ras A and B. Those proteins are expressed in a mutated form in at least a quarter of cancers with an even greater incidence for some histological types of tumour and according to the form of Ras. For example, mutations of K-Ras B are found in 80 to 90% of pancreatic carcinomas and 30 to 60% of colon cancers (Int. J. Oncol., 7, 1995, 413-421). Numerous preclinical data have demonstrated the role of that oncogene in tumour progression, more especially in cell growth phenomena. It is an essential link in the transmission of extracellular signals—such as those activated by growth factors—to diverse cytosolic kinases and then to the nucleus, for integration in terms of proliferation, cell death and cell survival (Cancer Met. Rev., 13, 1994, 67-89; Curr. Opin. Genetics & Develop., 8, 1998, 49-54; J. Biol. Chem. 273, 1998, 19925-19928), or of regulation with the tumour environment—angiogenesis in particular (Cancer Res., 55, 1995, 4575-4580).

The search for FTase inhibitors is thus of considerable interest in oncology (Curr. Opin. Chem. Biol., 2, 1998, 40-48). As 0.5% of animal proteins are probably prenylated and in the majority geranylgeranylated, specific inhibitors of FTase relative to the GGTases, and more especially GGTase-I, which is similar in structure to FTase, are of considerable interest. The first work with such inhibitors, peptidomimetic analogues of the famesylation consensus sequence, and the following work with molecules obtained by chemical library screening, confirmed the anti-tumour strategy in in vitro and animal experiments (Annu. Rev. Pharmacol. Toxicol., 37, 1997, 143-166; Biochim. Biophys. Acta, 1423, 1999, C19-C30; Cancer Res., 58, 1998, 4947-4956). Fibroblasts specially transfected with the mutated H-Ras protein gene and implanted in an animal develop a tumour mass the growth of which is reduced as a function of the dose of FTase inhibitor received by the animal. In the case of transgenic animals that express a mutated form of H-Ras under the control of an appropriate promoter causing the random appearance of spontaneous mammary or salivary tumours, those same inhibitors bring about the regression of established tumours and block the appearance of new ones for the duration of the treatment. Finally, such products are also active in reducing the growth of human xenotransplants in the mouse, with a possible effect of increasing survival, depending on the model. The mutated Ras protein is not the only indirect target of those inhibitors in tumour pathology (The Lancet Oncology, 2, 2001, 18-26; Cell. Mol. Life Sci., 58, 2001, 1636-1649). The study of multiple tumour models has enabled confirmation of inhibition of tumour growth independently of the presence of mutated Ras proteins. That effect could be partly associated with a direct antiangiogenic activity and thus could be independent of the oncogenic profile of the tumour (Eur. J. Cancer, 35, 1999, 1394-1401). This observation reinforces and increases the potential for anti-tumour use of that class of inhibitors, and the absence of debilitating side effects on normal cell functions is also favourable for the inhibition of FTase in any pathology associated with mechanisms changed or amplified by a farnesylated protein or by farnesylated proteins. Aside from cancer, this applies especially, for example, to restenosis following angioplasty or vascular surgery, and to type I neurofibromatosis (Mol. Cell. Biol., 17,1997, 862-872).

The compounds of the invention have a novel structure and are capable of selective inhibition of FTase relative to the GGTases. They will accordingly be useful in the treatment of all pathologies associated with intracellular signalling through Ras proteins or other farnesylated proteins, and in pathologies associated with angiogenesis amplification. They will thus be of use in the treatment of cancer, but also in the treatment of restenosis following angioplasty or vascular surgery, and in the treatment of type I neurofibromatosis.

### DETAILED DESCRIPTION OF THE INVENTION

The present invention concerns more especially the compounds of formula (I):

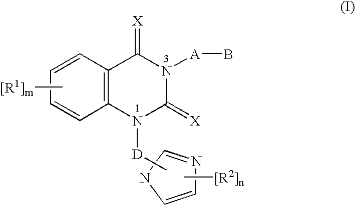

wherein:

A represents a bond, or an alkylene, alkenylene, alkynylene, T, *-A₁-T-, *-T-A₁-, *-A₁-T-A′₁- or *-A₁-T-A′₁-T′- group (wherein T and T′, which may be identical or different, each represents a carbonyl, carbonyloxy, thio, sulphinyl, sulphonyl, oxy, amino, aminoalkyl, aminoaryl, carbonylamino, carbonylaminoalkyl, carbonylaminoaryl, oxycarbonyl, aminocarbonyl, aminoalkylcarbonyl, aminoarylcarbonyl, sulphonylamino, sulphonylaminoalkyl, sulphonylaminoaryl, aminosulphonyl, aminoalkylsulphonyl or aminoarylsulphonyl group; A₁ and A′₁, which may be identical or different, each represents an alkylene, alkenylene or alkynylene group; and the symbol “*” represents the point of attachment to the nitrogen atom N³ of the quinazoline ring),

B represents an optionally substituted alkyl, optionally substituted aryl, optionally substituted heteroaryl, optionally substituted cycloalkyl, optionally substituted heterocycloalkyl, optionally substituted arylaminoaryl or optionally substituted arylalkylaryl group,

D represents an alkylene group in which a carbon atom of the hydrocarbon chain may be substituted by an optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl or optionally substituted cycloalkylalkyl group,

X represents an oxygen or sulphur atom,

R¹ represents a halogen atom, or an alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl or alkoxycarbonylamino group,

R² represents a hydrogen atom, or an alkyl, aryl, heteroaryl, cycloalkyl, heterocycloalkyl, arylalkyl, heteroarylalkyl, cycloalkylalkyl or heterocycloalkylalkyl group, it being possible for each of those groups to be optionally substituted by a substituent selected from a halogen atom or an alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl, alkoxycarbonylamino group, optionally substituted arylamino or optionally substituted arylalkyl,

m represents an integer of from 0 to 4 inclusive,

n represents an integer of from 0 to 3 inclusive,

to their enantiomers, diastereoisomers, and also addition salts thereof with a pharmaceutically acceptable acid or base,

wherein:

where m is greater than 1, the R¹ groups may be identical to or different from one another,

where n is greater than 1, the R² groups may be identical to or different from one another,

when the two nitrogen atoms of the imidazolyl group are substituted, the imidazolyl group becomes a cationic imidazolinium group,

the term “alkyl” denotes a linear or branched hydrocarbon chain containing from 1 to 6 carbon atoms,

the term “alkoxy” denotes a linear or branched alkyl-oxy group containing from 1 to 6 carbon atoms,

the term “alkylene” denotes a linear or branched divalent hydrocarbon chain containing from 1 to 6 carbon atoms,

the term “alkenylene” denotes a linear or branched divalent hydrocarbon chain containing from 1 to 3 double bonds and from 2 to 6 carbon atoms,

the term “alkynylene” denotes a linear or branched divalent hydrocarbon chain containing from 1 to 3 triple bonds and from 2 to 6 carbon atoms,

the term “cycloalkyl” denotes a saturated or partially saturated mono- or poly-cyclic group containing from 3 to 10 carbon atoms,

the term “heterocycloalkyl” denotes a saturated or partially unsaturated mono- or poly-cyclic group of from 5 to 7 ring members containing from 1 to 3 hetero atoms selected from nitrogen, oxygen and sulphur,

the term “aryl” denotes a phenyl, naphthyl or biphenyl group,

the term “heteroaryl” denotes a mono- or bi-cyclic group that is aromatic or contains at least one aromatic ring and that has from 5 to 11 ring members and contains from 1 to 3 hetero atoms selected from nitrogen, oxygen and sulphur,

the expression “optionally substituted” governing the term alkyl denotes that from one to three carbon atoms of the hydrocarbon chain may be substituted by one to three identical or different substituents selected from halogen atom or an alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl or alkoxycarbonylamino group,

the expression “optionally substituted” governing the terms aryl, heteroaryl, cycloalkyl, heterocycloalkyl, arylalkylaryl and arylaminoaryl denotes, unless specified to the contrary, that the cyclic moiety or moieties of those groups may be substituted by from one to three identical or different substituent selected from a halogen atom, or an alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl or alkoxycarbonylamino group,

the term “perhaloalkyl” denotes a methyl, ethyl, propyl or butyl group substituted by from 1 to 9 halogen atoms.

Of the cycloalkyl groups, mention may be made of the groups cyclopropyl, cyclobutyl, cyclopentyl, cyclohexyl, bicyclo[2,2,1]heptyl, adamantyl, . . .

Of the heteroaryl groups, mention may be made of the groups pyridyl, furyl, thienyl, indolyl, . . . .

Of the heterocycloalkyl groups, mention may be made of the groups piperidyl, piperazinyl, morpholino, . . . .

Among the pharmaceutically acceptable acids there may be mentioned hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, etc . . . .

Among the pharmaceutically acceptable bases there may be mentioned sodium hydroxide, potassium hydroxide, triethylamine, tert-butylamine, etc.

The invention relates more especially to compounds of formula (I) wherein A represents a bond, a sulphonyl group or an alkylene group.

Advantageously, the invention relates to compounds of formula (I) wherein B represents an optionally substituted aryl or optionally substituted heteroaryl group.

In the compounds of formula (I) m is preferably 0.

Preferred compounds of the invention are those wherein X represents an oxygen atom.

Preferred compounds of the invention are those wherein D represents an alkylene group substituted by an optionally substituted aryl or optionally substituted arylalkyl group.

Other preferred compounds of formula (I) are those wherein R² represents a hydrogen atom, an alkyl or optionally substituted arylalkyl group.

In the preferred compounds of formula (I), n is 0, 1 or 2.

The present invention relates more especially to compounds of formula (I) wherein m is 0, X represents an oxygen atom, A represents a bond, a sulphonyl group or an alkylene group, B represents an optionally substituted phenyl group, a benzylphenyl group, a pyridyl group, an anilinophenyl group or a thienyl group, D represents an alkylene group that is unsubstituted or substituted by an optionally substituted phenyl or optionally substituted phenylmethyl group, n is 0, 1 or 2 and R² represents an alkyl or optionally substituted arylalkyl group.

The present invention relates more especially to compounds of formula (I) wherein m is 0, X represents an oxygen atom, A represents a bond, a sulphonyl group or an alkylene group, B represents an optionally substituted phenyl group, a benzylphenyl group, a pyridyl group, an anilinophenyl group or a thienyl group, D represents an alkylene group that is unsubstituted or substituted by an optionally substituted phenyl or optionally substituted phenylmethyl group and that is attached to one of the nitrogen atoms of the imidazolyl, n is 0, 1 or 2 and R² represents an alkyl or optionally substituted arylalkyl group.

The invention relates most especially to the following compounds: tert-butyl 4-(1-[2-benzyl-3-(1H-imidazol-1-yl)propyl]-2,4-dioxo-1,4-dihydro-3(2H)-quinazolinyl)phenylcarbamate, 1-[2-benzyl-3-(1H-imidazol-1-yl)propyl]-3-(3-bromophenyl)-2,4(1H,3H)-quinazolinedione, 1-[2-benzyl-3-(1-methyl-1H-imidazol-5-yl)propyl]-3-phenyl-2,4(1H,3H)-quinazolinedione bistrifluoroacetate, 3-(3-bromophenyl)-1-{3-[1-(4-cyanobenzyl)-1H-imidazol-5-yl]propyl}-2,4(1H,3H)-quinazolinedione.

The present invention relates also to a process for the preparation of compounds of formula(I), which process is characterised in that there is used as starting material a compound of formula (II):

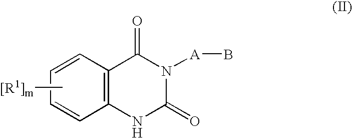

wherein R¹, A, B and m are as defined for formula (I), which is condensed with an alcohol compound of formula (III):

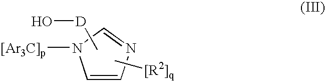

wherein Ar represents an optionally substituted phenyl group, q is an integer of from 0 to 2 inclusive, R² and D are as defined for formula (I) and p is an integer of from 0 to 1 inclusive,

to yield, in the presence of dialkyl azodicarboxylate and triarylphosphine, a compound of formula (IV):

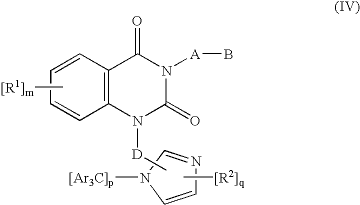

wherein Ar, R¹, R², A, B, D, m, p and q are as defined hereinbefore,

which is subjected to the action of a ketone-function thionation agent to yield a compound of formula (V):

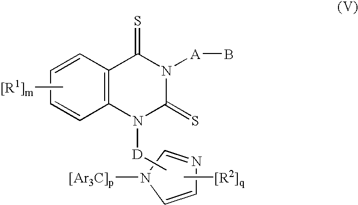

wherein Ar, R¹, R², A, B, D, m, p and q are as defined hereinbefore,

the compounds of formulae (IV) and (V) being represented by formula (VI):

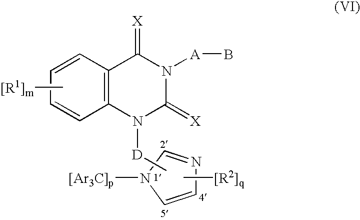

wherein Ar, R¹, R², A, B, D, m, p and q are as defined hereinbefore and X is as defined for formula (I),

which compound of formula (VI), when D is attached to the imidazolyl group by the nitrogen atom N_{1′} (where p is 0), corresponds to a compound of formula (I/a), a particular case of the compounds of formula (I):

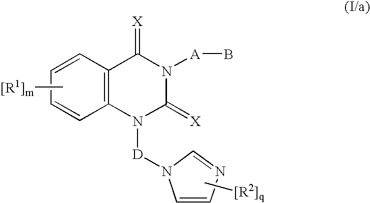

wherein R¹, R², A, B, D, X, m and q are as defined hereinbefore,

which may be subjected to the action of the following reagent: Hal-R³ wherein Hal represents a halogen atom and R³ may have any of the meanings of R² with the exception of an aryl or heteroaryl group,

to yield a cationic compound (I/b), a particular case of the compounds of formula (I):

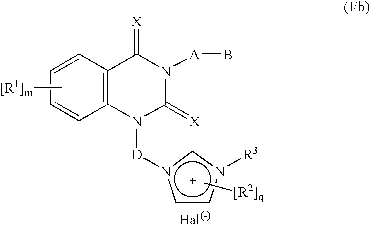

wherein R¹, R², R³, A, B, D, X, m and q are as defined hereinbefore and Hal⁽⁻⁾ represents the anion corresponding to the halogen atom Hal,

which compound of formula (VI), when D is attached to the imidazolyl group by the carbon atoms C_{2′}, C_{4′} and C_{5′} (where p is 1), corresponds to a compound of formula (VII):

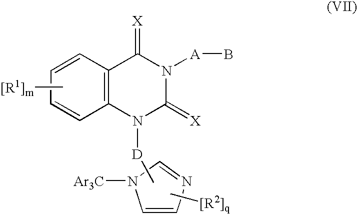

wherein Ar, R¹, R², A, B, D, X, m and q are as defined hereinbefore,

which may:

either be subjected to the action of an acid, such as hydrochloric acid for example, to obtain a deprotected compound (I/c), a particular case of the compounds of formula (I):

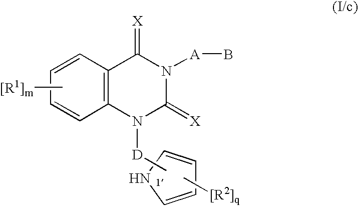

wherein R¹, R², A, B, D, X, m and q are as defined hereinbefore,

of which the nitrogen atom N_{1′} of the imidazolyl group may be substituted, optionally in the presence of an appropriate catalyst, by the following reagent: Hal-R⁴, wherein Hal is as defined hereinbefore and R⁴ may have any of the meanings of R² with the exception of a hydrogen atom and the groups aryl and heteroaryl, to yield, in alkaline medium, a compound of formula (I/d), a particular case of the compounds of formula (I):

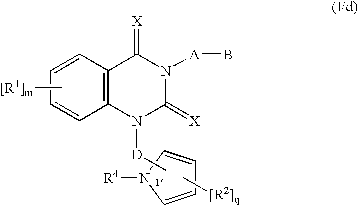

wherein R¹, R², R⁴, A, B, D, X, m and q are as defined hereinbefore,

or reacted with the following alkylating agent: Hal-R⁵, wherein Hal represents a halogen atom and R⁵ may have any of the meanings of R² with the exception of a hydrogen atom and the groups aryl and heteroaryl, to yield a cationic compound of formula (VIII):

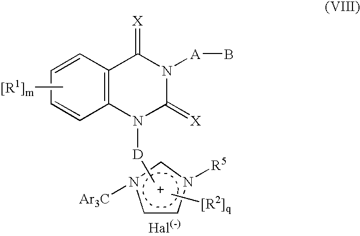

wherein Ar, R¹, R², R⁵, A, B, D, X, m, Hal⁽⁻⁾ and q are as defined hereinbefore,

which is deprotected, by heating in methanol, to yield a compound of formula (I/e), a particular case of the compounds of formula (I):

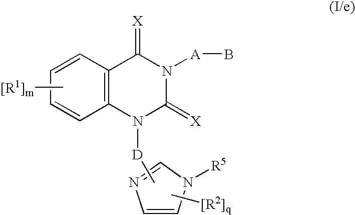

wherein R¹, R², R⁵, A, B, D, X, m and q are as defined hereinbefore,

the totality of the compounds of formulae (I/a) to (I/e) constituting the totality of the compounds of formula (I),

which may, if desired, be purified according to a conventional purification technique,

are optionally separated into their isomers (diastereoisomers, enantiomers and geometric isomers) by a conventional separation technique,

the intermediates of which may, if desired, be protected and deprotected in the course of the synthesis in order to facilitate access to the desired products,

which may, if desired, be converted into addition salts with a pharmaceutically acceptable acid or base.

The present invention relates also to pharmaceutical compositions comprising as active ingredient at least one compound of formula (I), in combination with one or more pharmaceutically acceptable, inert, non-toxic excipients or carriers.

Among the pharmaceutical compositions according to the invention there may be mentioned more especially those which are suitable for oral, parenteral, nasal or transdermal administration, tablets or dragees, sublingual tablets, gelatin capsules, lozenges, suppositories, creams, ointments, dermal gels, etc.

The useful dosage varies in accordance with the age and weight of the patient, the nature and severity of the disorder, and also the administration route, which may be oral, nasal, rectal or parenteral. In general, the unit dose ranges from 0.05 to 500 mg for a treatment of from 1 to 3 administrations per 24 hours.

The Examples which follow illustrate the invention and do not limit it in any way. The structures of the described compounds were confirmed by customary spectroscopic and spectrometric techniques.

The starting materials employed are either known products or are products prepared according to known procedures.

Preparation 1: 3-(3-Bromophenyl)-2,4(1H,3H)-quinazolinedione

A mixture of 10 g of isatoic anhydride, 6 g of 3-bromoaniline and 20 g of carbonyldiimidazole is stirred for 1 hour 30 minutes at 120° C. After returning to ambient temperature, 100 ml of water are added to the solid residue, and insoluble material is recovered by filtration and washed with a minimum amount of ethanol. The 15 g of crude solid obtained are purified by chromatography on silica gel (dichloromethane/ethyl acetate: 95/5). 10 g of the expected product are obtained.

Melting point: (cap.) >250° C.

Elemental Microanalysis:

| | C | H | N | Br |
|---|---|---|---|---|
| % Calculated: | 53.02 | 2.86 | 8.83 | 5.19 |
| % Found: | 52.73 | 2.84 | 8.60 | 4.90 |

Preparation 2: tert-Butyl 4-(2,4-dioxo-1,4-dihydro-3(2H)-quinazolinyl)phenylcarbamate

The product is obtained by proceeding as in Preparation 1, with the replacement of 3-bromoaniline by tert-butyl 4-anilinocarbamate.

Elemental Microanalysis:

| | C | H | N |
|---|---|---|---|
| % Calculated: | 64.58 | 5.42 | 11.89 |
| % Found: | 64.22 | 5.37 | 11.62 |

Preparation 3: 3-(Phenylsulphonyl)-2,4(1H,3H)-quinazolinedione

The product is obtained by proceeding in accordance with the procedure described in Patent Application WO 00/10982.

Preparation 4: 3-(3-Thienylsulphonyl)-2,4(1H,3H)-quinazolinedione

The product is obtained by proceeding as in Preparation 3, but starting from 3-thiophenesulphonamide.

Elemental Microanalysis:

| | C | H | N | S |
|---|---|---|---|---|
| % Calculated: | 46.74 | 2.62 | 9.09 | 20.80 |
| % Found: | 47.35 | 2.89 | 9.53 | 21.22 |

Preparation 5: 3-Methyl-2,4(1H,3H)-quinazolinedione

A solution of 6.6 g of triphosgene in 100 ml of dichloromethane is added in the course of 30 minutes to a solution of 10 g of 2-amino-N-methylbenzamide in 500 ml of dichloromethane. The reaction mixture is stirred at 60° C. for 1 hour, and the solvent is evaporated off. The 10.5 g of crude product obtained are purified by chromatography on silica gel (toluene/methanol/NH₃: 95/5/0.1). 2.2 g of product are obtained.

Melting point: (cap.) 205-209° C.

Elemental Microanalysis:

| | C | H | N |
|---|---|---|---|
| % Calculated: | 61.36 | 4.58 | 15.90 |
| % Found: | 61.66 | 4.38 | 15.00 |

Preparation 6: 3-(1H-Imidazol-1-yl)-1-propanol

100 ml of bromopropanol are added dropwise in the course of 1 hour to a suspension of 68 g of imidazole and 43 g of sodium hydroxide in 100 ml of butanol. The reaction mixture is stirred at 140° C. for ½ hour. After returning to ambient temperature, insoluble material is removed by filtration and washed with ethanol. After removal of the solvent by evaporation, the oil obtained is purified by chromatography on silica gel (dichloromethane/methanol/NH₃: 95/5/0.1). 95 g of product in the form of an oil are obtained.

Elemental Microanalysis:

| | C | H | N |
|---|---|---|---|
| % Calculated: | 57.12 | 7.94 | 22.20 |
| % Found: | 57.35 | 7.42 | 21.80 |

Preparation 7: 3-(1H-Imidazol-1-yl)-2-phenyl-1-propanol

Step a: Ethyl 3-(1H-imidazol-1-yl)-2-phenylpropanoate

10 g of imidazole are added to a solution of 10 g of ethyl atropate [Helvetica Chimica Acta, 69, 2048 (1986)] in 100 ml of ethanol. The reaction mixture is stirred at ambient temperature for 24 hours and the solvent is evaporated off under reduced pressure. The 20 g of oil obtained are purified by chromatography on silica gel (dichloromethane/methanol/NH₃: 95/5/0.1). 11 g of product in the form of an oil are obtained. methane/methanol/NH₃: 95/5/0.1). 11 g of product in the form of an oil are obtained.

Elemental Microanalysis:

| | C | H | N |
|---|---|---|---|
| % Calculated: | 68.83 | 6.60 | 11.47 |
| % Found: | 68.38 | 6.76 | 11.18 |

Step b 3-(1H-Imidazol-1-yl)-2-phenyl-1-propanol

2.2 g of lithium aluminium hydride (LiAlH₄) are added in the course of 30 minutes, under an inert atmosphere, to a solution of 11 g of ethyl 3-(1H-imidazol-1-yl)-2-phenylpropanoate in 300 ml of THF at 0° C. The reaction mixture is then stirred for 16 hours at ambient temperature. After hydrolysis with moist sodium sulphate (Na₂SO₄), insoluble material is removed by filtration. The solvent is evaporated off under reduced pressure, the residue is taken up in dichloromethane and washed with water and the organic phase is dried over magnesium sulphate. 6 g of product are obtained.

Elemental Microanalysis:

| | C | H | N |
|---|---|---|---|
| % Calculated: | 71.76 | 6.48 | 13.85 |
| % Found: | 72.67 | 7.00 | 12.49 |

Preparation 8: 4-[2-Hydroxy-1-(1H-imidazol-1-ylmethyl)ethyl]benzonitrile

2-(4-Bromophenyl)-3-imidazol-1-yl-1-propanol is prepared according to the same operating conditions as in Preparation 7.

5 g of zinc cyanide (ZnCN₂) and 1.5 g of tetrakis(triphenylphosphine)palladium (Pd(PPh₃)₄) are added to a solution of 5.5 g of 2-(4-bromophenyl)-3-imidazol-1-yl-1-propanol in 30 ml of degassed DMF. The reaction mixture is stirred for 15 minutes in a microwave at a power of 300 W. Insoluble material is removed by filtration. The 7.5 g of crude product recovered after removal of the solvent by evaporation are purified by chromatography on silica gel (dichloromethane/methanol/NH₃: 95/5/0.1). 4.2 g of product are obtained.

Melting point: (cap.) 140-143° C.

Elemental Microanalysis:

| | C | H | N |
|---|---|---|---|
| % Calculated: | 68.71 | 5.77 | 18.49 |
| % Found: | 67.95 | 5.58 | 18.10 |

Preparation 9: 2-Benzyl-3-(1H-imidazol-1-yl)-1-propanol

The product is obtained by proceeding as in Preparation 7, starting from ethyl 2-benzylacrylate [Synthetic Communication, 18, 11, 1213 (1988)] instead of ethyl atropate. 6.5 g of product are obtained in the form of an oil.

Elemental Microanalysis:

| | C | H | N |
|---|---|---|---|
| % Calculated: | 72.19 | 7.16 | 12.95 |
| % Found: | 71.85 | 7.01 | 12.65 |

Preparation 10: 3-(1H-Imidazol-1-yl)-1-phenyl-1-propanol

Step a: 3-(1H-Imidazol-1-yl)-1-phenyl-1-propanone

A suspension of 1.7 g of 3-chloropropiophenone, 0.1 g of potassium iodide (KI) and 2 g of imidazole is heated at 150° C. for 16 hours. After returning to ambient temperature, the product is purified by chromatography on silica gel (dichloromethane/ethanol: 98/2). 0.9 g of product is obtained.

Elemental Microanalysis:

| | C | H | N |
|---|---|---|---|
| % Calculated: | 71.98 | 6.04 | 13.99 |
| % Found: | 71.97 | 6.06 | 13.86 |

Step b: 3-(1H-Imidazol-1-yl)-1-phenyl-1-propanol

2 g of sodium borohydride are added in the course of 30 minutes to 4 g of compound prepared in the above Step dissolved in 60 ml of methanol. The reaction mixture is stirred for 2 hours at ambient temperature. After removal of the solvent by evaporation, the product is purified by chromatography on silica gel (dichloromethane/methanol: 95/5). 1.6 g of product is obtained.

Melting point: (cap.) 108-110° C.

Elemental Microanalysis:

| | C | H | N |
|---|---|---|---|
| % Calculated: | 71.26 | 6.98 | 13.85 |
| % Found: | 70.55 | 6.86 | 13.48 |

Preparation 11: 4-[1-Hydroxy-3-(1H-imidazol-1-yl)propyl]benzonitrile

1-(4-Bromophenyl)-3-imidazol-1-yl-1-propanol is synthesised as in Preparation 10 starting from 1-[(4-bromophenyl)-3-imidazol-1-yl]phenyl-1-propanone. The replacement of bromine by a cyano group is carried out under the same operating conditions as in Preparation 8. 4.5 g of product are obtained.

Melting point: (cap.) 94-97° C.

Elemental Microanalysis:

| | C | H | N |
|---|---|---|---|
| % Calculated: | 68.71 | 5.77 | 18.49 |
| % Found: | 67.83 | 5.45 | 18.02 |

Preparation 12: 3-(1H-Imidazol-1-yl)-3-phenyl-1-propanol

A suspension of 1.6 g of ethyl 3-(1H-imidazol-1-yl)-3-phenylacrylate [Tetrahedron Letters, 37, 40, 7249 (1996) and 0.6 g of 5% palladium on graphite (Pd/C) in 10 ml of ethanol is stirred under 60 psi of hydrogen, maintained for 24 hours at ambient temperature. The catalyst is removed by filtration. After removal of the solvent by evaporation, 1.1 g of ethyl 3-(1H-imidazol-1-yl)-3-phenylpropanoate is obtained. The latter is reduced under the same operating conditions as those used in Preparation 7. 0.4 g of product is obtained.

Melting point: (cap.) 63-66° C.

Elemental Microanalysis:

| | C | H | N |
|---|---|---|---|
| % Calculated: | 71.26 | 6.98 | 13.85 |
| % Found: | 70.82 | 6.51 | 13.63 |

Preparation 13: 3-(1-Trityl-1H-imidazol-2-yl)-1-propanol

Step a: Ethyl 3-(1H-imidazol-2-yl)acrylate

A suspension of 10.9 g of 1H-imidazole-2-carbaldehyde, 47 g of K₂CO₃ and 28 ml of ethyl triethoxyphosphonoacetate in 550 ml of ethanol is stirred at 70° C. for 1 hour. Insoluble material is removed by filtration and the solvent is evaporated off under reduced pressure. The oil obtained is purified by chromatography on silica gel (dichloromethane/methanol: 95/5). 12.7 g of product are obtained.

Elemental Microanalysis:

| | C | H | N |
|---|---|---|---|
| % Calculated: | 57.82 | 6.07 | 16.86 |
| % Found: | 57.61 | 6.06 | 16.87 |

Step b: Ethyl 3-(1H-imidazol-2-yl)propanoate

A suspension of 12.5 g of compound prepared in the above Step and 1.5 g of Pd/C (10%) in 500 ml of ethanol is stirred under 60 psi of hydrogen at ambient temperature for 16 hours. The catalyst is removed by filtration and the solvent is evaporated off under reduced pressure. 12.5 g of product are obtained.

Melting point: (cap.) 107-109° C.

Elemental Microanalysis:

| | C | H | N |
|---|---|---|---|
| % Calculated: | 57.13 | 7.19 | 16.66 |
| % Found: | 57.06 | 7.18 | 16.55 |

Step c: Ethyl 3-(1-trityl-H-imidazol-2-yl)propanoate

A solution of 8.4 g of compound prepared in the above Step, 15 ml of triethylamine and 15 g of trityl chloride in 50 ml of dimethylformamide (DMF) is stirred at ambient temperature for 16 hours. 150 ml of water are added to the reaction mixture. 21 g of product are recovered after filtration.

Melting point: (cap.) 222-225° C.

Elemental Microanalysis:

| | C | H | N |
|---|---|---|---|
| % Calculated: | 79.00 | 6.38 | 6.82 |
| % Found: | 79.06 | 6.42 | 6.44 |

Step d: 3-(1-Trityl-1H-imidazol-2-yl)-1-propanol

The ester prepared in the above Step is reduced with lithium aluminium hydride (LiAlH₄) under the same operating conditions as those used in Preparation 7.

Melting point: (cap.) 138-140° C.

Elemental Microanalysis:

| | C | H | N |
|---|---|---|---|
| % Calculated: | 81.49 | 6.56 | 7.60 |
| % Found: | 80.85 | 6.23 | 7.41 |

Preparation 14: 3-(1-Trityl-1H-imidazol-4-yl)-1-propanol

The compound is synthesised starting from methyl 3-(1H-imidazol-4-yl)acrylate in accordance with the procedures of Steps a-d described above in Preparation 13.

Elemental Microanalysis:

| | C | H | N |
|---|---|---|---|
| % Calculated: | 81.49 | 6.56 | 7.60 |
| % Found: | 81.26 | 6.59 | 6.55 |

Preparation 15: 3-(4-Chlorophenyl)-2,4(1H,3H)-quinazolinedione

The product is obtained by proceeding as in Preparation 1, with the replacement of 3-bromoanaline by 4-chloroaniline.

Preparation 16: 3-(2-Methoxyphenyl)-2,4(1H,3H)-quinazolinedione

The product is obtained by proceeding as in Preparation 1, with the replacement of 3-bromoanaline by 2-methoxyaniline.

Preparation 17: 3-(2-Fluorophenyl)2,4(1H,3H)-quinazolinedione

The product is obtained by proceeding as in Preparation 1, with the replacement of 3-bromoanaline by 2-fluoroaniline.

Preparation 18: 3-(2-Chlorophenyl)-2,4(1H,3H-quinazolinedione

The product is obtained by proceeding as in Preparation 1, with the replacement of 3-bromoanaline by 2-chloroaniline.

Preparation 19: 3-(2-Bromophenyl)-2,4(1H,3H)-quinazolinedione

The product is obtained by proceeding as in Preparation 1, with the replacement of 3-bromoanaline by 2-bromoaniline.

Preparation 20: 3-(2-Trifluoromethylphenyl)-2,4(1H,3H)-quinazolinedione

The product is obtained by proceeding as in Preparation 1, with the replacement of 3-bromoanaline by 2-trifluoromethylaniline.

Preparation 21: 3-(2-Nitrophenyl)-2,4(1H,3H)-quinazolinedione

The product is obtained by proceeding as in Preparation 1, with the replacement of 3-bromoanaline by 2-nitroaniline.

Preparation 22: 3-(2-Anilinophenyl)-2,4(1H,3H)-quinazolinedione

The product is obtained by proceeding as in Preparation 1, with the replacement of 3-bromoanaline by 2-anilinoaniline.

Preparation 23: 3-(2-Benzylphenyl)2,4(1H,3H)-quinazolinedione

The product is obtained by proceeding as in Preparation 1, with the replacement of 3-bromoanaline by 2-benzylaniline.

Preparation 24: 3-(3-Fluorophenyl)-2,4(1H,3H)-quinazolinedione

The product is obtained by proceeding as in Preparation 1, with the replacement of 3-bromoanaline by 3-fluoroaniline.

Preparation 25: 3-(3-Chlorophenyl)-2,4(1H,3H)-quinazolinedione

The product is obtained by proceeding as in Preparation 1, with the replacement of 3-bromoanaline by 3-chloroaniline.

Preparation 26: 3-(3-Trifluoromethylphenyl)-2,4(1H,3H)-quinazolinedione

The product is obtained by proceeding as in Preparation 1, with the replacement of 3-bromoanaline by 3-trifluoromethylaniline.

Preparation 27: 3-(3-Nitrophenyl)-2,4(1H,3H)-quinazolinedione

The product is obtained by proceeding as in Preparation 1, with the replacement of 3-bromoanaline by 3-nitroaniline.

Preparation 28: 3-(3-Cyanophenyl)-2,4(1H,3H)-quinazolinedione

The product is obtained by proceeding as in Preparation 1, with the replacement of 3-bromoanaline by 3-cyanoaniline.

Preparation 29: 3-(3-Methoxyphenyl)-2,4(1H,3H)-quinazolinedione

The product is obtained by proceeding as in Preparation 1, with the replacement of 3-bromoanaline by 3-methoxyaniline.

Preparation 30: 3-(3-Dimethylaminophenyl)-2,4(1H,3H)-quinazolinedione

The product is obtained by proceeding as in Preparation 1, with the replacement of 3-bromoanaline by 3-dimethylaminoaniline.

Preparation 31: 3-(3,5-dichlorophenyl)-2,4(1H,3H)-quinazolinedione

The product is obtained by proceeding as in Preparation 1, with the replacement of 3-bromoanaline by 3,5-dichloroaniline.

Preparation 32: 3-[3,5-Bis(trifluoromethyl)phenyl]-2,4(1H,3H)-quinazolinedione

The product is obtained by proceeding as in Preparation 1, with the replacement of 3-bromoanaline by 3,5-di(trifluoromethyl)aniline.

Preparation 33: 3-(2,3-Dimethylphenyl)-2,4(1H,3H)-quinazolinedione

The product is obtained by proceeding as in Preparation 1, with the replacement of 3-bromoanaline by 2,3-dimethylaniline.

Preparation 34: 3-(4-Fluorophenyl)-2,4(1H,3H)-quinazolinedione

The product is obtained by proceeding as in Preparation 1, with the replacement of 3-bromoanaline by 4-fluoroaniline.

Preparation 35: 3-(4-Bromophenyl)-2,4(1H,3H)-quinazolinedione

The product is obtained by proceeding as in Preparation 1, with the replacement of 3-bromoanaline by 4-bromoaniline.

Preparation 36: 3-(4-Trifluoromethylphenyl)-2,4(1H,3H)-quinazolinedione

The product is obtained by proceeding as in Preparation 1, with the replacement of 3-bromoanaline by 4-trifluoromethylaniline.

Preparation 37: 3-(4-Nitrophenyl)-2,4(1H,3H)-quinazolinedione

The product is obtained by proceeding as in Preparation 1, with the replacement of 3-bromoanaline by 4-nitroaniline.

Preparation 38: 3-(4-Cyanophenyl)-2,4(1H,3H)-quinazolinedione

The product is obtained by proceeding as in Preparation 1, with the replacement of 3-bromoanaline by 4-cyanoaniline.

Preparation 39: 3-(4-Methylphenyl)-2,4(1H,3H)-quinazolinedione

The product is obtained by proceeding as in Preparation 1, with the replacement of 3-bromoanaline by 4-methylaniline.

Preparation 40: 3-(4-Methoxyphenyl)-2,4(1H,3H-quinazolinedione

The product is obtained by proceeding as in Preparation 1, with the replacement of 3-bromoanaline by 4-methoxyaniline.

Preparation 41: 3-(4-Dimethylaminophenyl)-2,4(1H,3H)-quinazolinedione

The product is obtained by proceeding as in Preparation 1, with the replacement of 3-bromoanaline by 4-dimethylaminoaniline.

Preparation 42: 3-(4-Anilinophenyl)-2,4(1H,3H)-quinazolinedione

The product is obtained by proceeding as in Preparation 1, with the replacement of 3-bromoaniline by 4-anilinophenylamine.

Preparation 43: 3-(2-Pyridyl)-2,4(1H,3H)-quinazolinedione

The product is obtained by proceeding as in Preparation 1, with the replacement of 3-bromoanaline by 2-aminopyridine.

Preparation 44: 3-(3-Pyridyl)-2,4(1H,3H)-quinazolinedione

The product is obtained by proceeding as in Preparation 1, with the replacement of 3-bromoanaline by 3-aminopyridine.

Preparation 45: 3-(4-Pyridyl)-2,4(1H,3H)-quinazolinedione

The product is obtained by proceeding as in Preparation 1, with the replacement of 3-bromoanaline by 4-aminopyridine.

Preparation 46: 3-Cyclohexyl-2,4(1H,3H)-quinazolinedione

The product is obtained by proceeding as in Preparation 1, with the replacement of 3-bromoanaline by cyclohexaneamine.

Preparation 47: 3-Bicyclo[2.2.1]hept-2-yl-2,4(1H,3H)-quinazolinedione

The product is obtained by proceeding as in Preparation 1, with the replacement of 3-bromoanaline by bicyclo[2.2.1]hept-2-ylamine.

Preparation 48: 3-(1,3-Benzodioxol-5-yl)-2,4(1H,3H)-quinazolinedione

The product is obtained by proceeding as in Preparation 1, with the replacement of 3-bromoanaline by 1,3-benzodioxol-5-ylamine.

Preparation 49: 3-(1-Naphthyl)-2,4(1H,3H)-quinazolinedione

The product is obtained by proceeding as in Preparation 1, with the replacement of 3-bromoanaline by 1-naphthylamine.

Preparation 50: 3-Benzyl-2,4(1H,3H)-quinazolinedione

The product is obtained by proceeding as in Preparation 1, with the replacement of 3-bromoanaline by benzylamine.

Preparation 51: 3-(2-Phenethyl)-2,4(1H,3H)-quinazolinedione

The product is obtained by proceeding as in Preparation 1, with the replacement of 3-bromoanaline by 2-phenethylamine.

Preparation 52: 3-(2-Phenylpropyl)-2,4(1H,3H)-quinazolinedione

The product is obtained by proceeding as in Preparation 1, with the replacement of 3-bromoanaline by 2-phenylpropylamine.

Preparation 53: 3-(2-Phenylbutyl)-2,4(1H,3H)-quinazolinedione

The product is obtained by proceeding as in Preparation 1, with the replacement of 3-bromoanaline by 2-phenylbutylamine.

Preparation 54: 3-(2-Pyridylmethyl)-2,4(1H,3H)-quinazolinedione

The product is obtained by proceeding as in Preparation 1, with the replacement of 3-bromoanaline by 2-pyridylmethylamine.

Preparation 55: 3-(3-Pyridylmethyl)-2,4(1H,3H)-quinazolinedione

The product is obtained by proceeding as in Preparation 1, with the replacement of 3-bromoanaline by 3-pyridylmethylamine.

Preparation 56: 3-(Cyclopropylmethyl)-2,4(1H,3H)-quinazolinedione

The product is obtained by proceeding as in Preparation 1, with the replacement of 3-bromoanaline by cyclopropylmethylamine.

Preparation 57: 3-(1-Adamantylmethyl)-2,4(1H,3H)-quinazolinedione

The product is obtained by proceeding as in Preparation 1, with the replacement of 3-bromoanaline by 1-adamantylmethylamine.

Preparation 58: 3-(2-Oxo-2-phenethyl)-2,4(1H,3H)-quinazolinedione

The product is obtained by proceeding as in Preparation 1, with the replacement of 3-bromoanaline by 2-oxophenethylamine.

Preparation 59: 3-(3,4-Dichlorophenyl)-2,4(1H,3H)-quinazolinedione

The product is obtained by proceeding as in Preparation 1, with the replacement of 3-bromoanaline by 3,4-dichloroaniline.

Preparation 60: 3-(2-Cyanophenyl)-2,4(1H,3H)-quinazolinedione

The product is obtained by proceeding as in Preparation 1, with the replacement of 3-bromoanaline by 2-cyanoaniline.

Preparation 61: 2-(4-Bromobenzyl)-3-(1H-imidazol-1-yl)-1-propanol

Step a: Ethyl 2-(4-bromobenzyl)-3-(1H-imidazol-1-yl)propanoate

A suspension of 60 ml of ethyl benzoylacetate, 100 g of 4-bromobenzyl bromide, 84 g of potassium carbonate and 16 g of sodium iodide in 200 ml of THF is stirred for 4 hours at 60° C. 56 g of potassium carbonate, 20 g of paraformaldehyde and 400 ml of THF are added to the reaction mixture and the mixture is stirred for 20 hours at 60° C. The solvent is evaporated off. The product is extracted with ether. The 125 g of oil are taken up in 1 litre of ethanol and 125 g of imidazole are added to the solution. The mixture is stirred for 20 hours at 70° C. The solvent is evaporated off under reduced pressure. The product obtained is purified by chromatography on silica gel (dichloromethane/methanol-NH₃: 95/5). 208 g of product are obtained.

Step b: 2-(4-Bromobenzyl)-3-(1H-imidazol-1-yl)-1-propanol

6.5 g of lithium aluminium hydride (LiAlH₄) are added in the course of 30 minutes to a solution of the compound of the above Step a in 800 ml of ether. The reaction mixture is stirred for 2 hours at 25° C. 50 g of moist sodium sulphate are added to the reaction mixture in the course of 20 minutes. Insoluble material is removed by filtration. The solvent is evaporated off under reduced pressure. 23 g of product are obtained by crystallisation from ether.

Preparation 62: 2-(4-Cyanobenzyl)-3-(1H-imidazol-1-yl)-1-propanol

A suspension of 5 g of the compound of Preparation 61, 4.5 g of Zn(CN)₂ and 1.2 g of Pd(PPh₃)₄ in 25 ml of dimethylformamide is stirred for 15 minutes under microwaves. Insoluble material is removed by filtration. The product is purified by chromatography on silica gel (dichloromethane/methanol: 95/5). 4.5 g of product are obtained.

### EXAMPLE 1

### 1-[3-(1H-Imidazol-1-yl)propyl]-3-phenyl-2,4(1H,3H)-quinazolinedione Fumarate

A solution of 1.5 ml of diethyl azodicarboxylate in 10 ml of THF is added dropwise to a suspension of 2 g of the commercial product 3-phenyl-2,4(1H,3H)-quinazolinedione, 1.2 g of the product of Preparation 6 and 5 g of triphenylphosphine on resin in 100 ml of tetrahydrofuran (THF). The reaction mixture is stirred for 16 hours, and insoluble material is removed by filtration. The 5 g of product recovered after removal of the solvent by evaporation are purified by chromatography on silica gel (toluene/methanol: 95/5), and the product obtained is converted into its fumarate salt by the addition of fumaric acid in ethanol.

Melting point: (cap.) 174-176° C.

Elemental Microanalysis:

| | C | H | N |
|---|---|---|---|
| % Calculated: | 62.33 | 4.79 | 12.11 |
| % Found: | 61.60 | 4.81 | 11.63 |

### EXAMPLE 2

### tert-Butyl 4-(1-[2-benzyl-3-(1H-imidazol-1-yl)propyl]-2,4-dioxo-1,4-dihydro-3(2H)-quinazolinyl)phenylcarbamate

The experimental protocol is identical to that of Example 1, using instead of 3-phenyl-2,4(1H,3H)-quinazolinedione the compound of Preparation 2 and instead of the product of Preparation 6 that of Preparation 9.

Elemental Microanalysis:

| | C | H | N |
|---|---|---|---|
| % Calculated: | 69.67 | 6.03 | 12.70 |
| % Found: | 68.49 | 6.22 | 11.98 |

Separation of the enantiomers of Example 2 was carried out on a chiral column, enabling the two enantiomers (R) and (S) to be obtained in a more than 99% optically pure form:

Enantiomer 1:

Melting point: (cap.) decomposition >150° C.

Elemental Microanalysis:

| | C | H | N |
|---|---|---|---|
| % Calculated: | 69.67 | 6.03 | 12.70 |
| % Found: | 68.56 | 5.89 | 12.19 |

Enantiomer 2:

Melting point: (cap.) decomposition >150° C.

Elemental Microanalysis:

| | C | H | N |
|---|---|---|---|
| % Calculated: | 69.67 | 6.03 | 12.70 |
| % Found: | 68.38 | 6.09 | 12.33 |

### EXAMPLE 3

### 1-[2-Benzyl-3-(1H-imidazol-1-yl)propyl]-3-(3-bromophenyl)-2,4(1H,3H)-quinazolinedione

The experimental protocol is identical to that of Example 1, using instead of 3-phenyl-2,4(1H,3H)-quinazolinedione the compound of Preparation 1 and instead of the product of Preparation 6 that of Preparation 9.

Elemental Microanalysis:

| | C | H | N | Br |
|---|---|---|---|---|
| % Calculated: | 62.92 | 4.50 | 10.87 | 15.50 |
| % Found: | 62.31 | 4.72 | 10.34 | 15.21 |

### EXAMPLE 4

### 1-[3-(1H-Imidazol-1-yl)-2-phenylpropyl]-3-(phenylsulphonyl)-2,4(1H,3H)-quinazolinedione

The experimental protocol is identical to that of Example 1, using instead of 3-phenyl-2,4(1H,3H)-quinazolinedione the compound of Preparation 3 and instead of the product of Preparation 6 that of Preparation 7.

Elemental Microanalysis:

| | C | H | N | S |
|---|---|---|---|---|
| % Calculated: | 64.18 | 4.56 | 11.52 | 6.59 |
| % Found: | 64.04 | 4.68 | 11.12 | 6.38 |

### EXAMPLE 5

### 1-[3-(1H-Imidazol-1-yl)-2-phenylpropyl]-3-(3-thienylsulphonyl)-2,4(1H,3H)-quinazolinedione

The experimental protocol is identical to that of Example 1, using instead of 3-phenyl-2,4(1H,3H)-quinazolinedione the compound of Preparation 4 and instead of the product of Preparation 6 that of Preparation 7.

Elemental Microanalysis:

| | C | H | N | S |
|---|---|---|---|---|
| % Calculated: | 58.52 | 4.09 | 11.37 | 13.02 |
| % Found: | 58.44 | 4.47 | 10.96 | 13.18 |

### EXAMPLE 6

### tert-Butyl 4-(1-[2-(4-cyanophenyl)-3-(1H-imidazol-1-yl)propyl]-2,4-dioxo-1,4-dihydro-3(2H)-quinazolinyl)phenylcarbamate

The experimental protocol is identical to that of Example 1, using instead of 3-phenyl-2,4(1H,3H)-quinazolinedione the compound of Preparation 2 and instead of the product of Preparation 6 that of Preparation 8.

Melting point: (cap.) decomposition >220° C.

Elemental Microanalysis:

| | C | H | N |
|---|---|---|---|
| % Calculated: | 68.31 | 5.37 | 14.94 |
| % Found: | 67.92 | 5.46 | 14.64 |

### EXAMPLE 7

### tert-Butyl 4-(1-[1-(4-cyanophenyl)-3-(1H-imidazol-1-yl)propyl]-2,4-dioxo-1,4-dihydro-3(2H)-quinazolinyl)phenylcarbamate

The experimental protocol is identical to that of Example 1, using instead of 3-phenyl-2,4(1H,3H1)-quinazolinedione the compound of Preparation 2 and instead of the product of Preparation 6 that of Preparation 11.

Elemental Microanalysis:

| | C | H | N |
|---|---|---|---|
| % Calculated: | 68.31 | 5.37 | 14.94 |
| % Found: | 67.94 | 5.46 | 14.51 |

### EXAMPLE 8

### 1-[2-Benzyl-3-(1H-imidazol-1-yl)propyl]-3-phenyl-2,4(1H,3H)-quinazolinedione

The experimental protocol is identical to that of Example 1, using instead of the product of Preparation 6 that of Preparation 9.

Elemental Microanalysis:

| | C | H | N |
|---|---|---|---|
| % Calculated: | 74.29 | 5.54 | 12.84 |
| % Found: | 73.49 | 6.30 | 10.89 |

Separation of the enantiomers of Example 8 was carried out on a chiral column enabling the two enantiomers (R) and (S) to be obtained in a more than 99% optically pure form:

Enantiomer 1:

| | C | H | N |
|---|---|---|---|
| % Calculated: | 74.29 | 5.54 | 12.84 |
| % Found: | 73.20 | 5.85 | 12.25 |

Enantiomer 2:

| | C | H | N |
|---|---|---|---|
| % Calculated: | 74.29 | 5.54 | 12.84 |
| % Found: | 73.10 | 5.41 | 12.25 |

### EXAMPLE 9

### tert-Butyl 4-(1-[3-(1H-imidazol-1-yl)-2-phenylpropyl]-2,4-dioxo-1,4-dihydro-3(2H)-quinazolinyl)phenylcarbamate

The experimental protocol is identical to that of Example 1, using instead of 3-phenyl-2,4(1H,3H)-quinazolinedione the compound of Preparation 2 and instead of the product of Preparation 6 that of Preparation 7.

Melting point: (cap.) 178-191° C.

Elemental Microanalysis:

| | C | H | N |
|---|---|---|---|
| % Calculated: | 69.26 | 5.81 | 13.03 |
| % Found: | 67.46 | 5.88 | 12.11 |

### EXAMPLE 10

### 3-(3-Bromophenyl)-1-[3-(1H-imidazol-1-yl)-2-phenylpropyl]-2,4(1H,3H)-quinazolinedione

The experimental protocol is identical to that of Example 1, using instead of 3-phenyl-2,4(1H,3H)-quinazolinedione the compound of Preparation 1 and instead of the product of Preparation 6 that of Preparation 7.

Elemental Microanalysis:

| | C | H | N | Br |
|---|---|---|---|---|
| % Calculated: | 62.29 | 4.22 | 11.17 | 15.94 |
| % Found: | 63.00 | 4.22 | 10.80 | 15.54 |

### EXAMPLE 11

### 4-[1-(3-(3-Bromophenyl)-2,4-dioxo-3,4-dihydro-1(2H)-quinazolinyl)-3-(1H-imidazol-1-yl)propyl]benzonitrile

The experimental protocol is identical to that of Example 1, using instead of 3-phenyl-2,4(1H,3H)-quinazolinedione the compound of Preparation 1 and instead of the product of Preparation 6 that of Preparation 11.

Elemental Microanalysis:

| | C | H | N | Br |
|---|---|---|---|---|
| % Calculated: | 61.61 | 3.83 | 13.30 | 15.18 |
| % Found: | 61.13 | 4.20 | 12.65 | 14.65 |

### EXAMPLE 12

### 1-[3-(1H-Imidazol-1-yl)-1-phenylpropyl]-3-phenyl-2,4(1H,3H)-quinazolinedione

The experimental protocol is identical to that of Example 1, using instead of the product of Preparation 6 that of Preparation 10.

Melting point: >230° C.

Elemental Microanalysis:

| | C | H | N |
|---|---|---|---|
| % Calculated: | 73.92 | 5.25 | 13.26 |
| % Found: | 73.47 | 5.43 | 13.02 |

### EXAMPLE 13

### 1-[3-(1H-Imidazol-1-yl)-3-phenylpropyl]-3-phenyl-2,4(1H,3H)-quinazolinedione

The experimental protocol is identical to that of Example 1, using instead of the product of Preparation 6 that of Preparation 12.

Melting point: (cap.) 202-205° C.

Elemental Microanalysis:

| | C | H | N |
|---|---|---|---|
| % Calculated: | 73.92 | 5.25 | 13.26 |
| % Found: | 73.69 | 5.36 | 13.29 |

### EXAMPLE 14

### 3-(3,4-Dichlorophenyl)-1-[3-(1H-imidazol-1-yl)propyl]-2,4(1H,3H)-quinazolinedione

The experimental protocol is identical to that of Example 1, using the compound of Preparation 59 instead of 3-phenyl-2,4(1H,3H)-quinazolinedione.

Melting point: (cap.) 188-192° C.

Elemental Microanalysis:

| | C | H | N | Cl |
|---|---|---|---|---|
| % Calculated: | 57.85 | 3.88 | 13.49 | 17.07 |
| % Found: | 57.56 | 4.10 | 13.07 | 16.76 |

### EXAMPLE 15

### 1-[3-(1H-Imidazol-1-yl)-2-phenylpropyl]-3-phenyl-2,4(1H,3H)-quinazolinedione

The experimental protocol is identical to that of Example 1, using instead of the product of Preparation 6 that of Preparation 7.

Melting point: (cap.) 111-115° C.

Elemental Microanalysis:

| | C | H | N |
|---|---|---|---|
| % Calculated: | 73.92 | 5.25 | 13.26 |
| % Found: | 73.52 | 5.49 | 12.39 |

Separation of the enantiomers of Example 15 was carried out on a chiral column enabling the two enantiomers (R) and (S) to be obtained in a more than 98% optically pure form:

Enantiomer 1:

Melting point: (cap.) 132-135° C.

Elemental Microanalysis:

| | C | H | N |
|---|---|---|---|
| % Calculated: | 73.92 | 5.25 | 13.26 |
| % Found: | 73.31 | 5.16 | 13.00 |

Enantiomer 2:

Melting point: (cap.) 133-136° C.

Elemental Microanalysis:

| | C | H | N |
|---|---|---|---|
| % Calculated: | 73.92 | 5.25 | 13.26 |
| % Found: | 72.72 | 5.12 | 12.88 |

### EXAMPLE 16

### 1-[3-(1H-Imidazol-1-yl)propyl]-3-(2-methoxyphenyl)-2,4(1H,3H)-quinazolinedione

The experimental protocol is identical to that of Example 1, using the compound of Preparation 16 instead of 3-phenyl-2,4(1H,3H)-quinazolinedione.

Elemental Microanalysis:

| | C | H | N |
|---|---|---|---|
| % Calculated: | 67.01 | 5.36 | 14.88 |
| % Found: | 65.55 | 5.42 | 14.53 |

### EXAMPLE 17

### tert-Butyl 4-(1-[3-(1H-imidazol-1-yl)propyl]-2,4-dioxo-1,4-dihydro-3(2H)-quinazolinyl)phenylcarbamate

The experimental protocol is identical to that of Example 1, using the compound of Preparation 2 instead of 3-phenyl-2,4(1H,3H)-quinazolinedione.

Melting point: (cap.) decomposition >228° C.

Elemental Microanalysis:

| | C | H | N |
|---|---|---|---|
| % Calculated: | 65.06 | 5.90 | 15.17 |
| % Found: | 63.04 | 5.89 | 14.51 |

### EXAMPLE 18

### 3-(3-Bromophenyl)-1-[3-(1H-imidazol-1-yl)propyl]-2,4(1H,3H)-quinazolinedione

The experimental protocol is identical to that of Example 1, using the compound of Preparation 1 instead of 3-phenyl-2,4(1H,3H)-quinazolinedione.

Elemental Microanalysis:

| | C | H | N | Br |
|---|---|---|---|---|
| % Calculated: | 56.48 | 4.03 | 13.17 | 18.79 |
| % Found: | 55.64 | 4.13 | 12.57 | 18.26 |

### EXAMPLE 19

### 1-[3-(1H-Imidazol-1-yl)propyl]-3-methyl-2,4(1H,3H)-quinazolinedione Fumarate

The experimental protocol is identical to that of Example 1, using the compound of Preparation 5 instead of 3-phenyl-2,4(1H,3H)-quinazolinedione.

Melting point: (cap.) 212-213° C.

Elemental Microanalysis:

| | C | H | N |
|---|---|---|---|
| % Calculated: | 54.54 | 5.30 | 13.39 |
| % Found: | 53.92 | 5.17 | 13.02 |

### EXAMPLE 20

### 3-(2,3-Dimethylphenyl)-1-[3-(1H-imidazol-1-yl)propyl]-2,4(1H,3H)-quinazolinedione

The experimental protocol is identical to that of Example 1, using the compound of Preparation 33 instead of 3-phenyl-2,4(1H,3H)-quinazolinedione.

Mass spectrometry: m/z=375.1

### EXAMPLE 21

### 1-[3-(1H-Imidazol4-yl)propyl]-3-phenyl-2,4(1H,3H)-quinazolinedione Hydrochloride

Step a: 3-Phenyl-1-[3-(1-trityl-H-imidazol-4-yl)propyl]-2,4(1H,3H)-quinazolinedione

The compound was synthesised according to the same procedure as that in Example 1 starting from the commercial product 3-phenyl-2,4(1H,3H)-quinazolinedione and the product of Preparation 14.

Step b: 1-[3-(1H-Imidazol-4-yl)propyl]-3-phenyl-2,4(1H,3H)-quinazolinedione

2 ml of 4N hydrochloric acid are added to a solution of 300 mg of the compound prepared in the above Step in 3 ml of methanol. The reaction mixture is stirred for 16 hours at 50° C. The solvent is evaporated off under reduced pressure, the product obtained is crystallised from an ethanol/ether mixture and 180 mg of product are isolated.

Melting point: (cap.) 235-238° C.

Elemental Microanalysis:

| | C | H | N | Cl |
|---|---|---|---|---|
| % Calculated: | 62.74 | 5.01 | 14.64 | 9.26 |
| % Found: | 62.12 | 4.97 | 14.62 | 9.95 |

### EXAMPLE 22

### 1-[3-(1-Benzyl-1H-imidazol-4-yl)propyl]-3-phenyl-2,4(1H,3H)-quinazolinedione

60 μl of benzyl bromide are added to a suspension of 150 mg of the compound prepared in Example 21 and 100 mg of potassium carbonate (K₂CO₃) in 2 ml of acetonitrile. The reaction mixture is stirred at ambient temperature for 16 hours. Insoluble material is removed by filtration; after evaporation of the solvent the product is purified by chromatography on silica gel (toluene/methanol: 97/3) in order to isolate 50 mg of product.

Elemental Microanalysis:

| | C | H | N |
|---|---|---|---|
| % Calculated: | 74.29 | 5.54 | 12.84 |
| % Found: | 74.13 | 6.04 | 11.68 |

### EXAMPLE 22′

### 1-[3-(1-Benzyl-1H-imidazol-5-yl)propyl]-3-phenyl-2,4-(1H,3H)-quinazolinedione

Step a: 3-Phenyl-1-[3-(1-trityl-1H-imidazol-4-yl)propyl]-2,4(1H,3H)-quinazolinedione

The compound was synthesised according to the same procedure as that in Example 1 starting from the commercial product 3-phenyl-2,4(1H,3H)-quinazolinedione and the product of Preparation 14.

Step b: 1-[3-(1-Benzyl-1H-imidazol-5-yl)propyl]-3-phenyl-2,4-(1H,3H)-quinazolinedione

80 μl of benzyl bromide are added to a solution of the compound synthesised in the above Step (300 mg) in 3 ml of methyl ethyl ketone. After having left the reaction mixture for 2 hours at 55° C. with stirring, the solvent is removed under reduced pressure. The solid residue is taken up in 3 ml of methanol and the mixture is heated at 70° C. for 2 hours. The solvent is then evaporated off and the crude solid is purified by chromatography on silica gel (dichloromethane/methanol: 95/5).

Elemental Microanalysis:

| | C | H | N |
|---|---|---|---|
| % Calculated: | 74.29 | 5.54 | 12.84 |
| % Found: | 74.42 | 5.94 | 11.85 |

### EXAMPLE 23

### 1-[3-(1-Methyl-1H-imidazol-4-yl)propyl]-3-phenyl-2,4(1H,3H)-quinazolinedione

The compound is prepared under the same operating conditions as for Example 22, using methyl iodide instead of benzyl bromide.

Elemental Microanalysis:

| | C | H | N |
|---|---|---|---|
| % Calculated: | 69.98 | 5.59 | 15.55 |
| % Found: | 69.25 | 5.87 | 15.58 |

### EXAMPLE 24

### 1-[3-(1H-Imidazol-2-yl)propyl]-3-phenyl-2,4(1H,3H)-quinazolinedione

Step a: 3-Phenyl-1-[3-(1-trityl-1H-imidazol-2-yl)propyl]-2,4(1H,3H)-quinazolinedione

The compound was synthesised according to the same procedure as that in Example 1 starting from the commercial product 3-phenyl-2,4(1H,3H)-quinazolinedione and the product of Preparation 13.

Step b: 1-[3-(1H-Imidazol-2-yl)propyl]-3-phenyl-2,4(1H,3H)-quinazolinedione

The compound is prepared under the same operating conditions as for Example 21, Step b, using the compound prepared in the above Step as starting reagent.

Melting point: (cap.) 230-232° C.

Elemental Microanalysis:

| | C | H | N |
|---|---|---|---|
| % Calculated: | 69.35 | 5.24 | 16.17 |
| % Found: | 68.89 | 5.24 | 15.94 |

### EXAMPLE 25

### 1-[3-(1-Methyl-1H-imidazol-2-yl)propyl]-3-phenyl-2,4(1H,3H)-quinazolinedione

The compound is prepared under the same operating conditions as for Example 23, using as starting material the compound of Example 24.

Melting point: (cap.) 232-235° C.

Elemental Microanalysis:

| | C | H | N |
|---|---|---|---|
| % Calculated: | 69.98 | 5.59 | 15.55 |
| % Found: | 70.09 | 5.60 | 15.34 |

### EXAMPLE 26

### 1-[3-(1-Benzyl-1H-imidazol-2-yl)propyl]-3-phenyl-2,4(1H,3H)-quinazolinedione

The compound is prepared under the same operating conditions as for Example 22, using as starting material the compound of Example 24.

Melting point: (cap.) 182-185° C.

Elemental Microanalysis:

| | | |
|---|---|---|
| C | H | N |

| | | | |
|---|---|---|---|
| % Calculated: | 74.29 | 5.54 | 12.84 |
| % Found: | 72.72 | 5.50 | 12.50 |

### EXAMPLE 27

### 2-[3-(2,4-Dioxo-3-phenyl-3,4-dihydro-1(2H)-quinazolinyl)propyl]-1,3-dimethyl-1H-imidazol-3-ium Iodide

The compound is prepared under the same operating conditions as for Example 25, using a large excess of methyl iodide.

Melting point: (cap.)>240° C.

Elemental Microanalysis:

| | C | H | N | I |
|---|---|---|---|---|
| % Calculated: | 52.60 | 4.61 | 11.15 | 25.96 |
| % Found: | 50.53 | 4.55 | 10.53 | 22.98 |

### EXAMPLE 28

### tert-Butyl 4-(1-[3-(1H-imidazol-5-yl)propyl]-2,4-dioxo-1,4-dihydro-3(2H)-quinazolinyl)phenylcarbamate

The compound is prepared under the same operating conditions as for Example 1, starting from the compound of Preparation 2 with the compound of Preparation 14 “detritylated” beforehand with hydrochloric acid.

Mass spectrometry: [M+H]⁺=462

### EXAMPLE 29

### tert-Butyl 4-(1-[3-(1-methyl-1H-imidazol-5-yl)propyl]-2,4-dioxo-1,4-dihydro-3(2H)-quinazolinyl)phenylcarbamate

The compound is prepared under the same operating conditions as for Example 22′, using the compounds of Preparations 2 and 14 as starting materials in Step a, and adding methyl iodide instead of benzyl bromide in Step b.

Mass spectrometry: [M+H]⁺=476

### EXAMPLE 30

### tert-Butyl 4-(1-[3-(1-benzyl-1H-imidazol-5-yl)propyl]-2,4-dioxo-1,4-dihydro-3(2H)-quinazolinyl)phenylcarbamate

The compound is prepared under the same operating conditions as for Example 22′, using the compounds of Preparations 2 and 14 as starting materials in Step a.

Mass spectrometry: [M+H]⁺=552

### EXAMPLE 31

### tert-Butyl 4-(1-{3-[1-(4-chlorobenzyl)-1H-imidazol-5-yl]propyl}-2,4-dioxo-1,4-dihydro-3(2H)-quinazolinyl)phenylcarbamate

The compound is prepared under the same operating conditions as for Example 30, using 1-bromomethyl-4-chlorobenzene as starting reagent instead of benzyl bromide.

Mass spectrometry: [M+H]⁺=586

### EXAMPLE 32

### tert-Butyl 4-(1-{3-[1-(4-cyanobenzyl)-1H-imidazol-5-yl]propyl}-2,4-dioxo-1,4-dihydro-3(2H)-quinazolinyl)phenylcarbamate

The compound is prepared under the same operating conditions as for Example 30, using 1-bromomethyl-4-cyanobenzene as starting reagent instead of benzyl bromide.

Mass spectrometry: [M+H]⁺=577

### EXAMPLE 33

### tert-Butyl 4-(1-{3-[1-(4-methoxybenzyl)-1H-imidazol-5-yl]propyl}-2,4-dioxo-1,4-dihydro-3(2H)-quinazolinyl)phenylcarbamate

The compound is prepared under the same operating conditions as for Example 30, using 1-chloromethyl-4-methoxybenzene as starting reagent instead of benzyl bromide.

Mass spectrometry: [M+H]⁺=582

### EXAMPLE 34

### tert-Butyl 4-(1-{3-[1-(4-methylbenzyl)-1H-imidazol-5-yl]propyl}-2,4-dioxo-1,4-dihydro-3(2H)-quinazolinyl)phenylcarbamate

The compound is prepared under the same operating conditions as for Example 30, using 1-bromomethyl-4-toluene as starting reagent instead of benzyl bromide.

Mass spectrometry: [M+H]⁺=566

### EXAMPLE 35

### 3-(4-Aminophenyl)-1-{3-[1-(4-methylbenzyl)-1H-imidazol-5-yl]-propyl}-2,4(1H,3H)-quinazolinedione

The compound is prepared starting from the product of Example 34 by conventional deprotection of the Boc group (hydrochloric acid in ethyl acetate).

### EXAMPLE 36

### 3-(4-Aminophenyl)-1-[3-(1H-imidazol-1-yl)propyl]-2,4(1H,3H)-quinazolinedione Dihydrochloride

The compound is prepared starting from the product of Example 17 by conventional deprotection of the Boc group (hydrochloric acid in ethyl acetate).

Melting point: (cap.) decomposition >230° C.

Elemental Microanalysis:

| | C | H | N | Cl |
|---|---|---|---|---|
| % Calculated: | 55.30 | 4.88 | 16.13 | 16.32 |
| % Found: | 55.68 | 4.53 | 15.90 | 16.98 |

### EXAMPLE 37

### 3-(4-Chlorophenyl)-1-[3-(1-methyl-1H-imidazol-5-yl)propyl]-2,4(1H,3H)-quinazolinedione

The compound is prepared under the same operating conditions as for Example 29, with the replacement of the compound of Preparation 2 by that of Preparation 15.

Mass spectrometry: [M+H]⁺=395

### EXAMPLE 38

### 1-[3-(1-Benzyl-1H-imidazol-5-yl)propyl]-3-(4-chlorophenyl)-2,4(1H,3H)-quinazolinedione

The compound is prepared under the same operating conditions as for Example 30, with the replacement of the compound of Preparation 2 by that of Preparation 15.

Mass spectrometry: [M+H]⁺=471

### EXAMPLE 39

### 3-(4-Chlorophenyl)-1-{3-[1-(4-methylbenzyl)-1H-imidazol-5-yl]-propyl}-2,4(1H,3H)-quinazolinedione

The compound is prepared under the same operating conditions as for Example 34, with the replacement of the compound of Preparation 2 by that of Preparation 15.

Mass spectrometry: [M+H]⁺=485

### EXAMPLE 40

### 3-(4-Chlorophenyl)-1-{3-[1-(4-methoxybenzyl)-1H-imidazol-5-yl]-propyl}-2,4(1H,3H)-quinazolinedione

The compound is prepared under the same operating conditions as for Example 33, with the replacement of the compound of Preparation 2 by that of Preparation 15.

Mass spectrometry: [M+H]⁺=501.1

### EXAMPLE 41

### 3-(4-Chlorophenyl)-1-{3-[1-(4-chlorobenzyl)-1H-imidazol-5-yl]-propyl}-2,4(1H,3H)-quinazolinedione

The compound is prepared under the same operating conditions as for Example 31, with the replacement of the compound of Preparation 2 by that of Preparation 15.

Mass spectrometry: [M+H]⁺=505

### EXAMPLE 42

### 3-(4-Chlorophenyl)-1-{3-[1-(4-cyanobenzyl)-1H-imidazol-5-yl]-propyl}-2,4(1H,3H)-quinazolinedione

The compound is prepared under the same operating conditions as for Example 32, with the replacement of the compound of Preparation 2 by that of Preparation 15.

Mass spectrometry: [M+H]⁺=496

### EXAMPLE 43

### 3-(3-Bromophenyl)-1-[3-(1H-imidazol-5-yl)propyl]-2,4(1H,3H)-quinazolinedione

The compound is prepared under the same operating conditions as for Example 21, starting in Step a from the compound of Preparation 1.

Mass spectrometry: [M+H]⁺=425

### EXAMPLE 44

### 3-(3-Bromophenyl)-1-[3-(1-methyl-1H-imidazol-5-yl)propyl]-2,4(1H,3H)-quinazolinedione

The compound is prepared under the same operating conditions as for Example 22′, using the compounds of Preparations 1 and 14 as starting materials in Step a, and adding methyl iodide instead of benzyl bromide in Step b.

Mass spectrometry: [M+H]⁺=439

### EXAMPLE 45

### 3-(3-Bromophenyl)-1-{3-[1-(4-methylbenzyl)-1H-imidazol-5-yl]-propyl}-2,4(1H,3H)-quinazolinedione

The compound is prepared under the same operating conditions as for Example 44, using 1-bromomethyl-4-toluene as starting reagent instead of methyl iodide.

Mass spectrometry: [M+H]⁺=529

### EXAMPLE 46

### 3-(3-Bromophenyl)-1-{3-[1-(4-chlorobenzyl)-1H-imidazol-5-yl]-propyl}-2,4(1H,3H)-quinazolinedione

The compound is prepared under the same operating conditions as for Example 44, using 1-bromomethyl-4-chlorobenzene instead of methyl iodide.

Mass spectrometry: [M+H]⁺=548.9

### EXAMPLE 47

### 3-(3-Bromophenyl)-1-{3-[1-(4-cyanobenzyl)-1H-imidazol-5-yl]-propyl}-2,4(1H,3H)-quinazolinedione

The compound is prepared under the same operating conditions as for Example 44, using 1-bromomethyl-4-cyanobenzene instead of methyl iodide.

Mass spectrometry: [M+H]⁺=540

### EXAMPLE 48

### 1-[3-(1-Benzyl-1H-imidazol-5-yl)propyl]-3-(3-bromophenyl)-2,4(1H,3H)-quinazolinedione

The compound is prepared under the same operating conditions as for Example 44, using benzyl bromide as starting reagent instead of methyl iodide.

Mass spectrometry: [M+H]⁺=515

### EXAMPLE 49

### 3-(3-Bromophenyl)-1-{3-[1-(4-methoxybenzyl)-1H-imidazol-5-yl]-propyl}-2,4(1H,3H)-quinazolinedione

The compound is prepared under the same operating conditions as for Example 44, using 1-chloromethyl-4-methoxybenzene as starting reagent instead of methyl iodide.

Mass spectrometry: [M+H]⁺=545.1

### EXAMPLE 50

### 3-(2-Methoxyphenyl)-1-[3-(1H-imidazol-5-yl)propyl]-2,4(1H,3H)-quinazolinedione

The compound is prepared under the same operating conditions as for Example 22′, using the compounds of Preparations 14 and 16 as starting materials in Step a, and proceeding in Step b as in Example 21, Step b.

Mass spectrometry: [M+H]⁺=377.1

### EXAMPLE 51

### 3-(2-Methoxyphenyl)-1-[3-(1-methyl-1H-imidazol-5-yl)propyl]-2,4(1H,3H)-quinazolinedione

The compound is prepared under the same operating conditions as for Example 22′, using the compounds of Preparations 14 and 16 as starting materials in Step a, and using methyl iodide instead of benzyl bromide in Step b.

Mass spectrometry: [M+H]⁺=391.1

### EXAMPLE 52

### 1-[3-(1-Benzyl-1H-imidazol-5-yl)propyl]-3-(2-methoxyphenyl)-2,4(1H,3H)-quinazolinedione

The compound is prepared under the same operating conditions as for Example 51, using benzyl bromide instead of methyl iodide.

Mass spectrometry: [M+H]⁺=466.7

### EXAMPLE 53

### 3-(2-Methoxyphenyl)-1-{3-[1-(4-methylbenzyl)-1H-imidazol-5-yl]-propyl}-2,4(1H,3H)-quinazolinedione

The compound is prepared under the same operating conditions as for Example 51, using 1-bromomethyl-4-toluene instead of methyl iodide.

Mass spectrometry: [M+H]⁺=481.1

### EXAMPLE 54

### 3-(2-Methoxyphenyl)-1-{3-[1-(4-methoxybenzyl)-1H-imidazol-5-yl]propyl}-2,4(1H,3H)-quinazolinedione

The compound is prepared under the same operating conditions as for Example 51, using 1-chloromethyl-4-methoxybenzene instead of methyl iodide.

Mass spectrometry: [M+H]⁺=497.1

### EXAMPLE 55

### 3-(2-Methoxyphenyl)-1-{3-[1-(4-chlorobenzyl)-1H-imidazol-5-yl]-propyl}-2,4(1H,3H)-quinazolinedione

The compound is prepared under the same operating conditions as for Example 51, using 1-bromomethyl-4-chlorobenzene instead of methyl iodide.

Mass spectrometry: [M+H]⁺=501.1

### EXAMPLE 56

### 3-(2-Methoxyphenyl)-1-{3-[1-(4-cyanobenzyl)-1H-imidazol-5-yl]-propyl}-2,4(1H,3H)-quinazolinedione

The compound is prepared under the same operating conditions as for Example 51, using 1-bromomethyl-4-cyanobenzene instead of methyl iodide.

Mass spectrometry: [M+H]⁺=492.1

### EXAMPLE 57

### 3-(4-Chlorophenyl)-1-[3-(1H-imidazol-5-yl)propyl]-2,4(1H,3H)-quinazolinedione

The compound is prepared under the same operating conditions as for Example 22′, using the compounds of Preparations 14 and 15 in Step a, and proceeding in Step b as in Example 21, Step b.

Mass spectrometry: [M+H]⁺=381

### EXAMPLE 58

### 1-{3-[1-(4-Methylbenzyl)-1H-imidazol-5-yl]propyl}-3-phenyl-2,4(1H,3H)-quinazolinedione

The compound is prepared under the same operating conditions as for Example 22′, using 1-bromomethyl-4-toluene instead of benzyl bromide in Step b.

Mass spectrometry: [M+H]⁺=451.1

### EXAMPLE 59

### 1-{3-[1-(4-Methoxybenzyl)-1H-imidazol-5-yl]propyl}-3-phenyl-2,4(1H,3H)-quinazolinedione

The compound is prepared under the same operating conditions as for Example 58, using 1-chloromethyl-4-methoxybenzene instead of 1-bromomethyl-4-toluene.

Mass spectrometry: [M+H]⁺=467.1

### EXAMPLE 60

### 1-{3-[1-(4-Chlorobenzyl)-1H-imidazol-5-yl]propyl}-3-phenyl-2,4(1H,3H)-quinazolinedione

The compound is prepared under the same operating conditions as for Example 58, using 1-bromomethyl-4-chlorobenzene instead of 1-bromomethyl-4-toluene.

Mass spectrometry: [M+H]⁺=471

### EXAMPLE 61

### 1-{3-[1-(4-Cyanobenzyl)-1H-imidazol-5-yl]propyl}-3-phenyl-2,4(1H,3H)-quinazolinedione

The compound is prepared under the same operating conditions as for Example 58, using 1-bromomethyl-4-cyanobenzene instead of 1-bromomethyl-4-toluene.

Mass spectrometry: [M+H]⁺=462.1

### EXAMPLE 62

### 3-(2-Fluorophenyl)-1-[3-(1H-imidazol-1-yl)propyl]-2,4(1H,3H)-quinazolinedione

The experimental protocol is identical to that of Example 1, using the compound of Preparation 17 instead of 3-phenyl-2,4(1H,3H)-quinazolinedione.

Mass spectrometry: [M+H]⁺=365.1

### EXAMPLE 63

### 3-(2-Chlorophenyl)-1-[3-(1H-imidazol-1-yl)propyl]-2,4(1H,3H)-quinazolinedione

The experimental protocol is identical to that of Example 1, using the compound of Preparation 18 instead of 3-phenyl-2,4(1H,3H)-quinazolinedione.

Mass spectrometry: [M+H]⁺=381.1

### EXAMPLE 64

### 3-(2-Bromophenyl)-1-[3-(1H-imidazol-1-yl)propyl]-2,4(1H,3H)-quinazolinedione

The experimental protocol is identical to that of Example 1, using the compound of Preparation 19 instead of 3-phenyl-2,4(1H,3H)-quinazolinedione.

Mass spectrometry: [M+H]⁺=425

### EXAMPLE 65

### 3-(2-Trifluoromethylphenyl)-1-[3-(1H-imidazol-1-yl)propyl]-2,4(1H,3H)-quinazolinedione

The experimental protocol is identical to that of Example 1, using the compound of Preparation 20 instead of 3-phenyl-2,4(1H,3H)-quinazolinedione.

Mass spectrometry: [M+H]⁺=415

### EXAMPLE 66

### 3-(2-Nitrophenyl)-1-[3-(1H-imidazol-1-yl)propyl]-2,4(1H,3H)-quinazolinedione

The experimental protocol is identical to that of Example 1, using the compound of Preparation 21 instead of 3-phenyl-2,4(1H,3H)-quinazolinedione.

Mass spectrometry: [M+H]⁺=392

### EXAMPLE 67

### 3-(2-Anilinophenyl)-1-[3-(1H-imidazol-1-yl)propyl]-2,4(1H,3H)-quinazolinedione

The experimental protocol is identical to that of Example 1, using the compound of Preparation 22 instead of 3-phenyl-2,4(1H,3H)-quinazolinedione.

Mass spectrometry: [M+H]⁺=438

### EXAMPLE 68

### 3-(2-Benzylphenyl)-1-[3-(1H-imidazol-1-yl)propyl]-2,4(1H,3H)-quinazolinedione

The experimental protocol is identical to that of Example 1, using the compound of Preparation 23 instead of 3-phenyl-2,4(1H,3H)-quinazolinedione.

Mass spectrometry: [M+H]⁺=437.1

### EXAMPLE 69

### 3-(2-Cyanophenyl)-1-[3-(1H-imidazol-1-yl)propyl]-2,4(1H,3H)-quinazolinedione

The experimental protocol is identical to that of Example 1, using the compound of Preparation 60 instead of 3-phenyl-2,4(1H,3H)-quinazolinedione.

Mass spectrometry: [M+H]⁺=372.1

### EXAMPLE 70

### 3-(3-Fluorophenyl)-1-[3-(1H-imidazol-1-yl)propyl]-2,4(1H,3H)-quinazolinedione

The experimental protocol is identical to that of Example 1, using the compound of Preparation 24 instead of 3-phenyl-2,4(1H,3H)-quinazolinedione.

Mass spectrometry: [M+H]⁺=365.1

### EXAMPLE 71

### 3-(3-Chlorophenyl)-1-[3-(1H-imidazol-1-yl)propyl]-2,4(1H,3H)-quinazolinedione

The experimental protocol is identical to that of Example 1, using the compound of Preparation 25 instead of 3-phenyl-2,4(1H,3H)-quinazolinedione.

Mass spectrometry: [M+H]⁺=381

### EXAMPLE 72

### 3-(3-Trifluoromethylphenyl)-1-[3-(1H-imidazol-1-yl)propyl]-2,4(1H,3H)-quinazolinedione

The experimental protocol is identical to that of Example 1, using the compound of Preparation 26 instead of 3-phenyl-2,4(1H,3H)-quinazolinedione.

Mass spectrometry: [M+H]⁺=415

### EXAMPLE 73

### 3-(3-Nitrophenyl)-1-[3-(1H-imidazol-1-yl)propyl]-2,4(1H,3H)-quinazolinedione

The experimental protocol is identical to that of Example 1, using the compound of Preparation 27 instead of 3-phenyl-2,4(1H,3H)-quinazolinedione.

Mass spectrometry: [M+H]⁺=392.1

### EXAMPLE 74

### 3-(3-Cyanophenyl)-1-[3-(1H-imidazol-1-yl)propyl]-2,4(1H,3H)-quinazolinedione

The experimental protocol is identical to that of Example 1, using the compound of Preparation 28 instead of 3-phenyl-2,4(1H,3H)-quinazolinedione.

Mass spectrometry: [M+H]⁺=372.1

### EXAMPLE 75

### 3-(3-Methoxyphenyl)-1-[3-(1H-imidazol-1-yl)propyl]-2,4(1H,3H)-quinazolinedione

The experimental protocol is identical to that of Example 1, using the compound of Preparation 29 instead of 3-phenyl-2,4(1H,3H)-quinazolinedione.

Mass spectrometry: [M+H]⁺=377.1

### EXAMPLE 76

### 3-(3-Dimethylaminophenyl)-1-[3-(1H-imidazol-1-yl)propyl]-2,4(1H,3H)-quinazolinedione

The experimental protocol is identical to that of Example 1, using the compound of Preparation 30 instead of 3-phenyl-2,4(1H,3H)-quinazolinedione.

Mass spectrometry: [M+H]⁺=390.1

### EXAMPLE 77

### 3-(3,5-Dichlorophenyl)-1-[3-(1H-imidazol-1-yl)propyl]-2,4(1H,3H)-quinazolinedione

The experimental protocol is identical to that of Example 1, using the compound of Preparation 31 instead of 3-phenyl-2,4(1H,3H)-quinazolinedione.

Mass spectrometry: [M+H]⁺=415

### EXAMPLE 78

### 3-[3,5-Bis(trifluoromethyl)phenyl]-1-[3-(1H-imidazol-1-yl)propyl]-2,4(1H,3H)-quinazolinedione

The experimental protocol is identical to that of Example 1, using the compound of Preparation 32 instead of 3-phenyl-2,4(1H,3H)-quinazolinedione.

Mass spectrometry: [M+H]⁺=483

### EXAMPLE 79

### 3-(4-Fluorophenyl)-1-[3-(1H-imidazol-1-yl)propyl]-2,4(1H,3H)-quinazolinedione

The experimental protocol is identical to that of Example 1, using the compound of Preparation 34 instead of 3-phenyl-2,4(1H,3H)-quinazolinedione.

Mass spectrometry: [M+H]⁺=365.1

### EXAMPLE 80

### 3-(4-Chlorophenyl)-1-[3-(1H-imidazol-1-yl)propyl]-2,4(1H,3H)-quinazolinedione

The experimental protocol is identical to that of Example 1, using the compound of Preparation 15 instead of 3-phenyl-2,4(1H,3H)-quinazolinedione.

Mass spectrometry: [M+H]⁺=381

### EXAMPLE 81

### 3-(4-Bromophenyl)-1-[3-(1H-imidazol-1-yl)propyl]-2,4(1H,3H)-quinazolinedione

The experimental protocol is identical to that of Example 1, using the compound of Preparation 35 instead of 3-phenyl-2,4(1H,3H)-quinazolinedione.

Mass spectrometry: [M+H]⁺=425

### EXAMPLE 82

### 3-(4-Trifluoromethylphenyl)-1-[3-(1H-imidazol-1-yl)propyl]-2,4(1H,3,H)-quinazolinedione

The experimental protocol is identical to that of Example 1, using the compound of Preparation 36 instead of 3-phenyl-2,4(1H,3H)-quinazolinedione.

Mass spectrometry: [M+H]⁺=415.1

### EXAMPLE 83

### 3-(4-Nitrophenyl)-1-[3-(1H-imidazol-1-yl)propyl]-2,4(1H,3H)-quinazolinedione

The experimental protocol is identical to that of Example 1, using the compound of Preparation 37 instead of 3-phenyl-2,4(1H,3H)-quinazolinedione.

Mass spectrometry: [M+H]⁺=392.1

### EXAMPLE 84

### 3-(4-Cyanophenyl)-1-[3-(1H-imidazol-1-yl)propyl]-2,4(1H,3H)-quinazolinedione

The experimental protocol is identical to that of Example 1, using the compound of Preparation 38 instead of 3-phenyl-2,4(1H,3H)-quinazolinedione.

Mass spectrometry: [M+H]⁺=372.1

### EXAMPLE 85

### 3-(4-Methylphenyl)-1-[3-(1H-imidazol-1-yl)propyl]-2,4(1H,3H)-quinazolinedione

The experimental protocol is identical to that of Example 1, using the compound of Preparation 39 instead of 3-phenyl-2,4(1H,3H)-quinazolinedione.

Mass spectrometry: [M+H]⁺=361

### EXAMPLE 86

### 3-(4-Methoxyphenyl)-1-[3-(1H-imidazol-1-yl)propyl]-2,4(1H,3H)-quinazolinedione

The experimental protocol is identical to that of Example 1, using the compound of Preparation 40 instead of 3-phenyl-2,4(1H,3H)-quinazolinedione.

Mass spectrometry: [M+H]⁺=377

### EXAMPLE 87

### 3-(4-Dimethylaminophenyl)1-[3-(1H-imidazol-1-yl)propyl]-2,4(1H,3H)-quinazolinedione

The experimental protocol is identical to that of Example 1, using the compound of Preparation 41 instead of 3-phenyl-2,4(1H,3H)-quinazolinedione.

Mass spectrometry: [M+H]⁺=390.1

### EXAMPLE 88

### 3-(4-Anilinophenyl)-1-[3-(1H-imidazol-1-yl)propyl]-2,4(1H,3H)-quinazolinedione

The experimental protocol is identical to that of Example 1, using the compound of Preparation 42 instead of 3-phenyl-2,4(1H,3H)-quinazolinedione.

Mass spectrometry: [M+H]⁺=438.1

### EXAMPLE 89

### 3-(2-Pyridyl)-1-[3-(1H-imidazol-1-yl)propyl]-2,4(1H,3H)-quinazolinedione

The experimental protocol is identical to that of Example 1, using the compound of Preparation 43 instead of 3-phenyl-2,4(1H,3H)-quinazolinedione.

Mass spectrometry: [M+H]⁺=348.1

### EXAMPLE 90

### 3-(3-Pyridyl)-1-[3-(1H-imidazol-1-yl)propyl]-2,4(1H,3H)-20-quinazolinedione

The experimental protocol is identical to that of Example 1, using the compound of Preparation 44 instead of 3-phenyl-2,4(1H,3H)-quinazolinedione.

Mass spectrometry: [M+H]⁺=348.1

### EXAMPLE 91

### 3-(4-Pyridyl)-1-[3-(1H-imidazol-1-yl)propyl]-2,4(1H,3H)-quinazolinedione

The experimental protocol is identical to that of Example 1, using the compound of Preparation 45 instead of 3-phenyl-2,4(1H,3H)-quinazolinedione.

Mass spectrometry: [M+H]⁺=348.1

### EXAMPLE 92

### 3-Cyclohexyl-1-[3-(1H-imidazol-1-yl)propyl]-2,4(1H,3H)-quinazolinedione

The experimental protocol is identical to that of Example 1, using the compound of Preparation 46 instead of 3-phenyl-2,4(1H,3H)-quinazolinedione.

Mass spectrometry: [M+H]⁺=353

### EXAMPLE 93

### 3-Bicyclo[2.2.1]hept-2-yl-1-[3-(1H-imidazol-1-yl)propyl]-2,4(1H,3H)-quinazolinedione

The experimental protocol is identical to that of Example 1, using the compound of Preparation 47 instead of 3-phenyl-2,4(1H,3H)-quinazolinedione.

Mass spectrometry: [M+H]⁺=365

### EXAMPLE 94

### 3-(1,3-Benzodioxol-5-yl)—1-[3-(1H-imidazol-1-yl)propyl]-2,4(1H,3H)-quinazolinedione

The experimental protocol is identical to that of Example 1, using the compound of Preparation 48 instead of 3-phenyl-2,4(1H,3 h)-quinazolinedione.

Mass spectrometry: [M+H]⁺=391.1

### EXAMPLE 95

### 3-(1-Naphthyl)-1-[3-(1H-imidazol-1-yl)propyl]-2,4(1H,3H)-quinazolinedione

The experimental protocol is identical to that of Example 1, using the compound of Preparation 49 instead of 3-phenyl-2,4(1H,3H)-quinazolinedione.

Mass spectrometry: [M+H]⁺=397.1

### EXAMPLE 96

### 3-Benzyl-1-[3-(1H-imidazol-1-yl)propyl]-2,4(1H,3H)-quinazolinedione

The experimental protocol is identical to that of Example 1, using the compound of Preparation 50 instead of 3-phenyl-2,4(1H,3H)-quinazolinedione.

Mass spectrometry: [M+H]⁺=361.1

### EXAMPLE 97

### 3-(2-Phenethyl)-1-[3-(1H-imidazol-1-yl)propyl]-2,4(1H,3H)-quinazolinedione

The experimental protocol is identical to that of Example 1, using the compound of Preparation 51 instead of 3-phenyl-2,4(1H,3H)-quinazolinedione.

Mass spectrometry: [M+H]⁺=375.2

### EXAMPLE 98

### 3-(3-Phenylpropyl)-1-[3-(1H-imidazol-1-yl)propyl]-2,4(1H,3H)-quinazolinedione

The experimental protocol is identical to that of Example 1, using the compound of Preparation 52 instead of 3-phenyl-2,4(1H,3H)-quinazolinedione.

Mass spectrometry: [M+H]⁺=389.2

### EXAMPLE 99

### 3-(4-Phenylbutyl)-1-[3-(1H-imidazol-1-yl)propyl]-2,4(1H,3H)-quinazolinedione

The experimental protocol is identical to that of Example 1, using the compound of Preparation 53 instead of 3-phenyl-2,4(1H,3H)-quinazolinedione.

Mass spectrometry: [M+H]⁺=403.3

### EXAMPLE 100

### 3-(2-Pyridylmethyl)-1-[3-(1H-imidazol-1-yl)propyl]-2,4(1H,3H)-quinazolinedione

The experimental protocol is identical to that of Example 1, using the compound of Preparation 54 instead of 3-phenyl-2,4(1H,3H)-quinazolinedione.

Mass spectrometry: [M+H]⁺=362.1

### EXAMPLE 101

### 3-(3-Pyridylmethyl)1-[3-(1H-imidazol-1-yl)propyl]-2,4(1H,3H)-quinazolinedione

The experimental protocol is identical to that of Example 1, using the compound of Preparation 55 instead of 3-phenyl-2,4(1H,3H)-quinazolinedione.

Mass spectrometry: [M+H]⁺=362.1

### EXAMPLE 102

### 3-(Cyclopropylmethyl)-1-[3-(1H-imidazol-1-yl)propyl]-2,4(1H,3H)-quinazolinedione

The experimental protocol is identical to that of Example 1, using the compound of Preparation 56 instead of 3-phenyl-2,4(1H,3H)-quinazolinedione.

Mass spectrometry: [M+H]⁺=325.2

### EXAMPLE 103

### 3-(Adamantylmethyl)-1-[3-(1H-imidazol-1-yl)propyl]-2,4(1H,3H)-quinazolinedione

The experimental protocol is identical to that of Example 1, using the compound of Preparation 57 instead of 3-phenyl-2,4(1H,3H)-quinazolinedione.

Mass spectrometry: [M+H]⁺=419

### EXAMPLE 104

### 3-(2-Oxo-2-phenethyl)-1-[3-(1H-imidazol-1-yl)propyl]-2,4(1H,3H)-quinazolinedione

The experimental protocol is identical to that of Example 1, using the compound of Preparation 58 instead of 3-phenyl-2,4(1H,311)-quinazolinedione.

Mass spectrometry: [M+H]⁺=389.1

### EXAMPLE 105

### 1-[2-Benzyl-3-(1-methyl-1H-imidazol-5-yl)propyl]-3-phenyl-2,4(1H,3H)-quinazolinedione Bistrifluoroacetate

The experimental protocol is identical to that of Example 21, using 2-benzyl-3-(1-trityl-1H-imidazol-5-yl)-1-propanol in Step a instead of the product of Preparation 14; the acidification in Step b was carried out with trifluoroacetic acid instead of hydrochloric acid followed by methylation of the imidazole under the same operating conditions as those of Example 23.

Elemental Microanalysis:

| | C | H | N |
|---|---|---|---|
| % Calculated: | 56.64 | 4.16 | 8.26 |
| % Found: | 55.11 | 4.15 | 7.94 |

### EXAMPLE 106

### 1-[2-Phenyl-3-(1-methyl-1H-imidazol-5-yl)propyl]-3-phenyl-2,4(1H,3H)-quinazolinedione

The experimental protocol is identical to that of Example 21, using 2-phenyl-3-(1-trityl-1H-imidazol-5-yl)-1-propanol in Step a instead of the product of Preparation 14, followed after Step b by methylation of the imidazole under the same operating conditions as those of Example 23.

Elemental Microanalysis:

| | C | H | N |
|---|---|---|---|
| % Calculated: | 74.29 | 5.54 | 12.84 |
| % Found: | 73.94 | 5.34 | 12.70 |

### EXAMPLE 107

### tert-Butyl 4-(1-[2-(4-bromobenzyl)-3-(1H-imidazol-1-yl)propyl]-2,4-dioxo-1,4-dihydro-3(2H)-quinazolinyl)phenylcarbamate

The experimental protocol is identical to that of Example 1, using instead of 3-phenyl-2,4-(1H,3H)-quinazolinedione the compound of Preparation 2 and instead of the product of Preparation 6 that of Preparation 61.

Mass spectrometry: [M+H]⁺=630

### EXAMPLE 108

### 4-[3-(3-Bromophenyl)-2,4-dioxo-3,4-dihydro-1 (2H)-quinazolinyl)-2-(1H-imidazol-1-ylmethyl)propyl]cyanophenyl

The experimental protocol is identical to that of Example 1, using instead of 3-phenyl-2,4(1H,3H)-quinazolinedione the compound of Preparation 1 and instead of the product of Preparation 6 that of Preparation 62.

Mass spectrometry: [M+H]⁺=540

### EXAMPLE 109

### tert-Butyl 4-(1-[2-(4-cyanobenzyl)-3-(1H-imidazol-1-yl)propyl]-2,4-dioxo-1,4-dihydro-3(2H)-quinazolinyl)phenylcarbamate

The experimental protocol is identical to that of Example 1, using instead of 3-phenyl-2,4(1H,3H)-quinazolinedione the compound of Preparation 2 and instead of the product of Preparation 6 that of Preparation 62.

Mass spectrometry: [M+H]⁺=577

Pharmacological Study

### EXAMPLE A

### Enzyme Tests

The two enzymes FTase and GGTase-I were purified starting from rat's brain. After grinding and centrifuging, the supernatant is precipitated with 30% ammonium sulphate and the resulting supernatant is subjected to another precipitation with 50% ammonium sulphate. The pellet is then passed through a column of phenyl agarose and the fractions collected after elution with sodium chloride are evaluated for their enzyme content in accordance with the “scintillation proximity assay” method described hereinbelow. The fractions corresponding to one or other of the two enzymes are then combined and frozen at −80° C. until use.

The determination of the enzymatic activity of the FTase is carried out in 96-well plates by a radioactive scintillation proximity assay method. The acceptor substrate composed of the carboxy terminal sequence of lamin B (YRASNRSCAIM) coupled to biotin is incubated in the presence of the radiolabelled donor substrate ([³H]farnesyl pyrophosphate), and of various concentrations of test compounds in dimethyl sulphoxide (DMSO). The reaction is initiated at 37° C. by adding FTase enzyme for a duration of one hour, and is then stopped with an appropriate buffer containing a suspension of beads impregnated with scintillant. Those beads are in addition coupled to streptavidin in order to capture, by coupling to biotin, the peptide susceptible to famesylation, and hence place the radiolabelled famesyl in contact with the scintillant. The plates are read in a radioactivity counter and the data are converted into percentages of a control in order to express the results in the form of the concentration of test product that causes 50% inhibition of famesylation (IC₅₀).

For GGTase-I an equivalent test was used, replacing the acceptor substrate with the biotinylated sequence TKCVIL and replacing the donor substrate with [³H]geranylgeranyl pyrophosphate.

Results:

The compounds of the present invention have IC₅₀s that are less than micromolar with respect to FTase, revealing their character as powerful inhibitors of that enzyme, and demonstrate an appreciable selectivity relative to GGTase-I, the IC₅₀s in that case being greater than micromolar.

By way of example, the compound of Example 2 has an IC₅₀ of 19 nM with respect to FTase.

### EXAMPLE B

### Cell Proliferation Tests

a) The RAT2 line of rat fibroblasts and an appropriate transfectant for the insertion of the gene v-H-ras were used to test the effectiveness of the claimed products on cells. The RAT2 cells allow the intrinsic toxicity of the test product to be characterised, while the transfected cells that exhibit a changed morphology and a more rapid growth rate serve to measure the desired specific effect on intracellular FTase.

The parental and transfected cells are cultured in 96-well plates for cell culture in the presence of medium containing 10% serum. Twenty four hours later, the test products are added to the same medium over a period of four days and the final quantity of cells is estimated indirectly by the cell viability method using 3-(4,5-dimethylthiazol-2-yl)-2,5-diphenyltetrazolium bromide (MTT).

Results:

In the case of the compounds of the invention, a slow-down in the growth of cells transfected with v-H-ras is observed in the range of some tens of nanomoles. That effect, reflecting the return of the transfected cells to the growth characteristics of the parental line, is accompanied also by a reversion of the morphology of the transfectants to the parental phenotype (spread, and loss of refraction). Several logarithmic units separate that specific effect from the cytotoxic effect observed on the RAT2 cells in the micromolar range, the most favourable differential being at least four units for the most active products.

By way of example, the compound of Example 2 has an IC₅₀ of inhibition of growth of cells transfected by the oncogene v-H-ras of 9 nM.

b) Additional tests on human carcinoma lines obtained from clinical biopsies are carried out. The lines used all come from the ATCC (American Type Culture Collection) and the test is carried out in 96-well plates for a duration of contact with the product corresponding to four doubling periods.

Results:

An indirect count by the MTT method allowed an anti-proliferative activity to be demonstrated with IC₅₀s of the micromolar order in the case of the compounds of the invention.

### EXAMPLE C

### Pharmaceutical Composition

Formulation for the preparation of 1000 tablets each containing a dose of
10 mg

| | |
|---|---|
| compound of Example 2 | 10 g |
| hydroxypropyl cellulose | 2 g |
| wheat starch | 10 g |
| lactose | 100 g |
| magnesium stearate | 3 g |
| talc | 3 g |

1- A compound selected from those of formula (I): 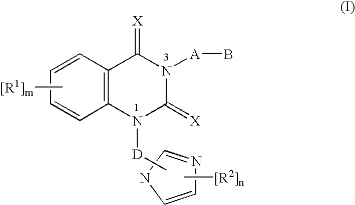
wherein:
A represents a bond, alkylene, alkenylene, alkynylene, T, *-A₁-T-, *-T-A₁-, *-A₁-T-A′₁- or *-A-T-A′₁-T′- (wherein T and T′, which may be identical or different, each represents carbonyl, carbonyloxy, thio, sulphinyl, sulphonyl, oxy, amino, aminoalkyl, aminoaryl, carbonylamino, carbonylaminoalkyl, carbonylaminoaryl, oxycarbonyl, aminocarbonyl, aminoalkylcarbonyl, aminoarylcarbonyl, sulphonylamino, sulphonylaminoalkyl, sulphonylaminoaryl, aminosulphonyl, aminoalkylsulphonyl or aminoarylsulphonyl; A₁ and A′₁, which may be identical or different, each represents alkylene, alkenylene or alkynylene; and the symbol “*” represents the point of attachment to the nitrogen atom N³ of the quinazoline ring),
B represents an optionally substituted alkyl, optionally substituted aryl, optionally substituted heteroaryl, optionally substituted cycloalkyl, optionally substituted heterocycloalkyl, optionally substituted arylaminoaryl or optionally substituted arylalkylaryl,
D represents alkylene in which a carbon atom of the hydrocarbon chain may be substituted by an optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl or optionally substituted cycloalkylalkyl,
X represents oxygen or sulphur,
R¹ represents halogen, alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl or alkoxycarbonylamino,
R² represents hydrogen, aryl, heteroaryl, cycloalkyl, heterocycloalkyl, arylalkyl, heteroarylalkyl, cycloalkylalkyl or heterocycloalkylalkyl, it being possible for each of those groups to be optionally substituted by a substituent selected from halogen, alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl, alkoxycarbonylamino, optionally substituted arylamino or optionally substituted arylalkyl,
m represents an integer of from 0 to 4 inclusive,
n represents an integer of from 0 to 3 inclusive,
its enantiomers, diastereoisomers, and addition salts thereof with a pharmaceutically acceptable acid or base,
wherein:
where m is greater than 1, R¹ may be identical to or different from one another,
where n is greater than 1, R² may be identical to or different from one another,
when the two nitrogen atoms of the imidazolyl group are substituted, the imidazolyl group becomes a cationic imidazolinium group,
the term “alkyl” denotes linear or branched hydrocarbon chain containing from 1 to 6 carbon,
the term “alkoxy” denotes linear or branched alkyl-oxy group containing from 1 to 6 carbon,
the term “alkylene” denotes linear or branched divalent hydrocarbon chain containing from 1 to 6 carbon,
the term “alkenylene” denotes linear or branched divalent hydrocarbon chain containing from 1 to 3 double bonds and from 2 to 6 carbon,
the term “alkynylene” denotes linear or branched divalent hydrocarbon chain containing from 1 to 3 triple bonds and from 2 to 6 carbon,
the term “cycloalkyl” denotes saturated or partially saturated mono- or poly-cyclic group containing from 3 to 10 carbon,
the term “heterocycloalkyl” denotes saturated or partially unsaturated mono- or poly-cyclic group of from 5 to 7 ring members containing from 1 to 3 hetero atoms selected from nitrogen, oxygen and sulphur,
the term “aryl” denotes phenyl, naphthyl or biphenyl,
the term “heteroaryl” denotes mono- or bi-cyclic group that is aromatic or contains at least one aromatic ring and that has from 5 to 11 ring members and contains from 1 to 3 hetero atoms selected from nitrogen, oxygen and sulphur,
the expression “optionally substituted” governing the term alkyl denotes that from one to three carbon of the hydrocarbon chain may be substituted by one to three identical or different substituents selected from halogen, alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl or alkoxycarbonylamino,
the expression “optionally substituted” governing the terms aryl, heteroaryl, cycloalkyl, heterocycloalkyl, arylalkylaryl and arylaminoaryl denotes, unless specified to the contrary, that the cyclic moiety or moieties of those groups may be substituted by from one to three identical or different substituent selected from halogen, alkyl, alkoxy, hydroxy, mercapto, cyano, amino, alkylamino, dialkylamino, nitro, perhaloalkyl, carboxy, alkoxycarbonyl, aminocarbonyl, alkylaminocarbonyl, dialkylaminocarbonyl, carbamoyl or alkoxycarbonylamino,
the term “perhaloalkyl” denotes methyl, ethyl, propyl or butyl substituted by from 1 to 9 halogen atoms.

2- A compound of claim 1, wherein A represents a bond, sulphonyl or alkylene.

3- A compound of claim 1, wherein B represents an optionally substituted aryl or optionally substituted heteroaryl.

4- A compound of claim 1, wherein m is 0.

5- A compound of claim 1, wherein X represents oxygen.

6- A compound of claim 1, wherein D represents alkylene substituted by an optionally substituted aryl or optionally substituted arylalkyl.

7- A compound of claim 1, wherein R² represents hydrogen, alkyl or optionally substituted arylalkyl.

8- A compound of claim 1, wherein n is 0, 1 or 2.

9- A compound of claim 1, wherein m is 0, X represents oxygen, A represents a bond, sulphonyl or alkylene, B represents optionally substituted phenyl, benzylphenyl, pyridyl, anilinophenyl or thienyl, D represents alkylene that is unsubstituted or substituted by optionally substituted phenyl or optionally substituted phenylmethyl, n is 0, 1 or 2 and R² represents alkyl or optionally substituted arylalkyl.

10- A compound of claim 1, wherein m is 0, X represents oxygen, A represents a bond, sulphonyl or alkylene, B represents optionally substituted phenyl, benzylphenyl, pyridyl, anilinophenyl or thienyl, D represents alkylene that is unsubstituted or substituted by optionally substituted phenyl or optionally substituted phenylmethyl and that is attached to one of the nitrogen atoms of the imidazolyl, n is 0, 1 or 2 and R² represents an alkyl or optionally substituted arylalkyl.

11- A compound of claim 1 which is tert-butyl 4-(1-[2-benzyl-3-(1H-imidazol-1-yl)propyl]-2,4-dioxo-1,4-dihydro-3(2H)-quinazolinyl)phenylcarbamate.

12- A compound of claim 1 which is 1-[2-benzyl-3-(1H-imidazol-1-yl)propyl]-3-(3-bromophenyl)-2,4(1H,3H)-quinazolinedione.

13- A compound of claim 1 which is 3-(3-bromophenyl)-1-{3-[1-(4-cyanobenzyl)-1H-imidazol-5-yl]propyl}-2,4(1H,3H)-quinazolinedione.

14- A compound of claim 1 which is 1-[2-benzyl-3-(1-methyl-1H-imidazol-5-yl)propyl]-3-phenyl-2,4(1H,3H)-quinazolinedione bistrifluoroacetate.

15- A method for treating a living animal body afflicted with cancer comprising the step of administering to the living animal body an amount of compound of claim 1 which is effective for alleviation of said cancer.

16- A method for treating a living animal body afflicted with restenosis or type I neurofibromatosis comprising the step of administering to the living animal body an amount of compound of claim 1 which is effective for alleviation of said conditions.

17- A pharmaceutical composition useful in treating cancer comprising as active principle an effective amount of a compound as claimed in claim 1, together with one or more pharmaceutical acceptable excipients or vehicles.

18- A pharmaceutical composition useful in the claim 16 method comprising as active principle an effective amount of a compound as claimed in claim 1, together with one or more pharmaceutical acceptable excipients or vehicle.
